(19) United States
(12) Patent Application Publication
(10) Pub. No.: US 2017/0362618 A1
(43) Pub. Date: Dec. 21, 2017

(54) HIGH SOLIDS ENZYMATIC HYDROLYSIS AND FERMENTATION OF PRETREATED BIOMASS

(71) Applicant: Abengoa Bioenergy New Technologies, LLC, Chesterfield, MO (US)

(72) Inventor: Quang A. Nguyen, Chesterfield, MO (US)

(21) Appl. No.: 15/534,840

(22) PCT Filed: Dec. 11, 2015
(86) PCT No.: PCT/US2015/065400

§ 371 Date: Jun. 9, 2017

Related U.S. Application Data
(60) Provisional application No. 62/091,109, filed on Dec. 12, 2014.

(51) Int. Cl. C12P 19/02; D21C 1/04; C12P 19/14; D21C 1/02; D21C 5/00; D21C 1/06

(52) U.S. Cl. CPC C12P 19/02; D21C 5/005; D21C 1/06; C12P 19/14; D21C 1/02; D21C 1/04; C12P 2201/00

(57) ABSTRACT

The present disclosure generally relates to a methods and systems for conversion of chemically pretreated lignocellulosic biomass to monosaccharides by enzymatic hydrolysis at high total solids concentration to provide for increased throughput and reduced enzyme usage in commercial scale processes.

### CROSS REFERENCE TO RELATED APPLICATION

The application claims the priority of U.S. Patent Application Ser. No. 62/091,109 filed on Dec. 12, 2014 which is hereby incorporated by reference in its entirety

### BACKGROUND

The field of the disclosure relates generally commercial scale preparation of monosaccharides and fermentation products from cellulosic biomass comprising lignocellulose, referred to herein as “lignocellulosic biomass”.

Biofuels, such as ethanol, are useful as an additive or replacement for petroleum-based fuels such as gasoline. Commercially, ethanol is typically produced by fermentation of sugars extracted from sugar cane or sugar beet or generated from sources of starch (e.g., corn starch) and/or from lignocellulosic biomass.

Lignocellulose is a complex structure comprising cellulose, hemicellulose, and lignin in which cellulose and hemicellulose are bound to the lignin. Cellulose is a polymer of D-glucose with β[1-4] linkages between each of the about 500 to 10,000 glucose units. Hemicellulose is a polymer of sugars, primarily D-xylose (in herbaceous biomass and hardwood) with other pentoses and some hexoses with β[1-4] linkages. Lignin is a complex random polyphenolic polymer. Fermentable monosaccharides are generated from the cellulosic and hemicellulosic lignocellulose components by chemical hydrolysis, thermochemical hydrolysis, enzymatic hydrolysis, or a combination thereof. Typical lignocellulosic biomass sources include, for example, corn stover, agricultural residues (e.g., straw, corn cobs, etc.), woody materials, energy crops (e.g., sorghum, poplar, etc.), and bagasse (e.g., sugarcane).

Commercially practical fermentation methods for preparing ethanol from lignocellulosic biomass require pretreatment steps for increasing the accessibility and bioavailability of cellulose and lignocellulose to downstream enzymatic hydrolysis where fermentable simple sugars (i.e., hexose and pentose monosaccharides) are generated. Lignocellulosic biomass pretreatment methods typically involve: (1) biomass impregnation with a water, a dilute acid or a dilute base; (2) contact of the impregnated biomass with steam under conditions of elevated temperature (e.g., about 150° C. to about 210° C.) and pressure (e.g., about 60 psig to about 250 psig); (3) rapid depressurization (“steam explosion”) resulting in condensate flashing and a physio-chemical separation of cellulose and hemicellulose from lignin, hydrolysis of β[1-4] linkages, and the liberation of lignocellulosic hydrolyzate comprising pentose monosaccharides, glucan (glucose polysaccharides) and cellulose. In such processes, lignocellulosic hydrolyzate typically has a solids content of from about 35 percent by weight (“wt.”) to about 50 wt. % and a temperature of about 100° C. to about 120° C. (wherein the temperature drop from the steam contact temperature is primarily due to heat of vaporization). Lignocellulosic hydrolyzate is generally cooled to a temperature of less than about 60° C. and the pH is adjusted to from about 4 to about 5.5 for enzymatic hydrolysis. Cooling is generally done by addition of chilled aqueous streams wherein a solids content of less than about 20 wt. % results. The adjusted lignocellulosic hydrolyzate is contacted with an enzyme cocktail comprising cellulase to generate saccharified lignocellulosic hydrolyzate comprising fermentable glucose monosaccharide. The saccharified lignocellulosic hydrolyzate may then be contacted with a source of an ethanolic organism to form ethanol by fermentation.

In commercial scale processes, such as having a lignocellulosic biomass throughput on the order of about 500 tons per day, or more, the total solids content in saccharifaction and fermentation is generally limited to no more than 20 wt. % because of inefficiencies related to cooling capacity and pH adjustment associated with such a solids content. More particularly, currently, cooling and pH control of hot lignocellulosic hydrolyzates generated from steam explosion of acid impregnated lignocellulosic biomass (such as having a temperature greater than about 100° C.) is achieved by mixing the hydrolyzate with chilled alkali solution in a high solid mixer, such as a pug mill. However, at high lignocellulosic hydrolyzate temperature and throughput, the volume of chilled water (at a practical lower temperature limit of about 10° C.) required to achieve temperatures suitable for enzymatic hydrolysis results in lignocellulosic hydrolysis dilution to a solids concentration of no more than 20 wt. %. Adequate cooling typically cannot be achieved at higher solids concentrations in excess of about 20 wt. %. Mixing at solids concentrations higher than 20 wt. % is further limiting because such lignocellulosic hydrolyzates are rheologically characterized by high viscosity (such as at least about 20,000 cP, and up to about 100,000 cP) resulting in poor flowability and poor mixability. Consequently, cooling lignocellulosic hydrolyzates at a concentration in excess of about 20 wt. % solids by current commercial methods results in material having non-homogeneous pH and non-homogeneous temperature such that, upon contact with an enzyme, enzyme denaturization occurs, high enzyme loading is required, and low yield based on cellulose occurs.

Still further, lignocellulosic biomass pretreatment liberates enzymatic and fermentation inhibitors such as aliphatic carboxylic acids (e.g., formic acid and acetic acid), furans (e.g., furfural and hydroxymethylfurfural) and phenolic compounds (e.g., 4-hydroxybenzoic acid, syringic acid, vanillic acid, ferulic acid, catechol, vanillin, 4-hydroxybenzaldehyde and syringaldehyde). Ethanol yield in commercial scale processes may be increased by removing or inactivating at least a portion of the lignocellulosic hydrolyzate inhibitors. However, the rheological characteristics of lignocellulosic hydrolyzates comprising in excess of 20 wt. % total solids limits the efficiency of and applicability of purification methods.

Significant throughput increases in lignocellulosic ethanol facilities and reduced enzyme usage could be realized if saccharification and fermentation could be done at a total solids loading of at least 25 wt. %. A need therefore exists for lignocellulosic pretreatment methods that provide for a homogeneous cooled, pH adjusted and purified lignocellulosic hydrolyzate having a total solids content of at least 25 wt. %.

### BRIEF DESCRIPTION

In one aspect of the present disclosure, a method for hydrolyzing lignocellulosic biomass is provided. The method comprises contacting the lignocellulosic biomass with an aqueous acid to form an acid impregnated lignocellulosic biomass having a pH of less than 4 and a total solids content of from about 30 percent by weight to about 70 percent by weight. The acid impregnated lignocellulosic biomass is contacted with steam to achieve a temperature of from about 150° C. to about 250° C. and a pressure of from about 55 psig (about 376 kPa gauge) to about 560 psig (about 3870 kPa gauge). The acid impregnated lignocellulosic biomass is depressurized to a pressure of from about 20 kPa to about 100 kPa gauge and to reduce the temperature of from about 105° C. to about 120° C. to form steam pretreated acid impregnated lignocellulosic biomass and flashed condensate. The steam pretreated acid impregnated biomass is extracted with a sufficient amount of a chilled aqueous alkaline solution to form (i) an extracted lignocellulosic biomass slurry comprising no greater than 20 percent by weight total solids and having a temperature of from 30° C. to about 70° C. and a pH of from about 4 to about 6 and (ii) a rich liquor aqueous stream comprising at least 60 percent by weight of the soluble compounds present in the stream pretreated acid impregnated biomass. The extracted lignocellulosic biomass slurry is dewatered to form (i) an extracted dewatered lignocellulosic biomass comprising at least 25 percent by weight total solids and having a substantially uniform pH of from about 4 to about 6 and a substantially uniform temperature of from 30° C. to about 70° C. and (ii) a lean liquor aqueous stream. Finally, the extracted dewatered lignocellulosic biomass is contacted with a first source of enzymes comprising at least cellulase to form a slurry having at least 25 percent by weight solids and hydrolyzing said slurry to form a primary hydrolyzate having a viscosity of less than about 10,000 centipoise and comprising hexose and pentose sugars.

In another aspect of the present disclosure, a method for hydrolyzing lignocellulosic biomass is provided. The method comprises contacting the lignocellulosic biomass with an aqueous acid to form an acid impregnated lignocellulosic biomass having a pH of less than 4 and a total solids content of from about 40 percent by weight to about 70 percent by weight. The acid impregnated lignocellulosic biomass is contacted with steam to achieve a temperature of from about 150° C. to about 250° C. and a pressure of from about 55 psig (about 378 kPa gauge) to about 560 psig (about 3870 kPa gauge). The acid impregnated lignocellulosic biomass is depressurized in a flash tank to a pressure of from about 20 kPa to about 35 kPa gauge to reduce the temperature to from about 105° C. to about 108° C. to form steam pretreated acid impregnated lignocellulosic biomass and flashed condensate. Alkaline chilled cooling water is combined with the steam pretreated acid impregnated lignocellulosic biomass in the flash tank and discharging the flash contents to a dynamic mixer. Additional alkaline chilled cooling water is added to the dynamic mixer and admixed with the alkaline chilled cooling water and the steam pretreated acid impregnated lignocellulosic biomass in the dynamic mixer to produce adjusted steam pretreated acid impregnated lignocellulosic biomass, wherein the amount and pH of the alkaline chilled cooling water is sufficient to provide an adjusted steam pretreated acid impregnated lignocellulosic biomass having a total solid content of at least 30 percent by weight, an average temperature of from about 30° C. to about 70° C., an average pH of from about 4 to about 6, and a viscosity of greater than about 20,000 centipoise. The adjusted steam pretreated acid impregnated lignocellulosic biomass is contacted with a first source of enzymes comprising at least cellulase in a primary enzymatic hydrolysis step to form a primary hydrolyzate having a viscosity of less than about 10,000 centipoise and comprising hexose and pentose sugars, and wherein the total solids content of the adjusted steam pretreated acid impregnated lignocellulosic biomass after contact with the first source of enzymes at least 25 percent by weight or from 25 percent by weight to less than 30 percent by weight.

### BRIEF DESCRIPTION OF THE DRAWINGS

FIG. 1 is a process flow diagram of a first aspect of the present disclosure.

FIG. 2 is a process flow diagram of a second aspect of the present disclosure.

FIG. 3 is a process flow diagram of a third aspect of the present disclosure.

FIG. 4 is a process flow diagram of a fourth aspect of the present disclosure.

FIG. 5 is a process flow diagram of a fifth aspect of the present disclosure.

FIG. 6 is a process flow diagram of a sixth aspect of the present disclosure.

FIG. 7 is a process flow diagram of a seventh aspect of the present disclosure.

FIG. 8 is a process flow diagram of a eighth aspect of the present disclosure.

FIG. 9 is a process flow diagram of a ninth aspect of the present disclosure.

FIG. 10 is a process flow diagram of a tenth aspect of the present disclosure.

### DETAILED DESCRIPTION

The present disclosure generally relates to a method for enzymatic hydrolysis of pretreated lignocellulosic biomass at high total solids concentration to provide for increased throughput and reduced enzyme usage in commercial scale processes.

Commercial scale processes for preparing monosaccharides and fermentation products from lignocellulosic biomass by steam explosion and enzymatic hydrolysis are typically limited to enzymatic hydrolysis at solids concentrations of no more than about 20 wt. % because of inefficiencies related to a cooling capacity required to reduce the temperature of the stream explosion hydrolyzate from greater than 100° C. to less than about 60° C. as required for enzymatic hydrolysis and the rheological characteristics of high viscosity and poor mixability associated with solids concentrations in excess of about 20 wt. % that render difficult achieving a homogeneous pH of from about 4 to about 6 by addition of acid or base as required for enzymatic hydrolysis. Further, the concentration of enzyme and fermentation inhibitors that are generated during hydrolysis of lignocellulosic biomass during steam explosion hydrolysis may be sufficiently high at a total solids loading in excess of about 20 wt. % to result in significantly reduced monosaccharide and/or fermentation product yield.

In accordance with the present disclosure, it has been discovered that steam exploded acid- or alkali-impregnated lignocellulosic biomass may be contacted with a chilled aqueous and pH-adjusted stream in an extraction unit operation at a total solids content of less than 25 wt. % followed by dewatering to a solids content of at least 25 wt. % to provide for a steam exploded lignocellulosic biomass stream having an essentially homogeneous temperature of less than 60° C. and an essentially homogeneous pH of from about 4 to about 6. In some aspects of the disclosure, the dewatered stream exploded lignocellulosic biomass is purified to reduce the level of inhibitors. The at least 25 wt. % high solids content stream may be contacted with a source of enzymes comprising a cellulase and a hemicellulase (xylanase) to provide for a hydrolyzate comprising hexose monosaccharides, pentose monosaccharides and, in some aspects, reduced inhibitor concentration. In some aspects of the disclosure, said hydrolyzate may be combined with a second source of enzymes comprising a glucoamylase to generate additional hexose monosaccharides and contact thereof with a source of a fermentation organism (e.g., yeast) to generate a fermentation product (e.g., ethanol). In some other aspects of the disclosure, said hydrolyzate may fractionated to form a monosaccharide stream and a solids steam.

In any of the various aspects of the present disclosure, increased throughput is provided in commercial scale lignocellulosic processing facilities by virtue of improved steam exploded lignocellulosic biomass cooling efficiency, improved steam exploded lignocellulosic biomass pH adjustment efficiency, improved cellulose and hemicellulose conversion because of reduced inhibitor concentration, and higher throughput through existing enzymatic hydrolysis and fermentation equipment resulting from increased solids content. It is believed that improvement over existing processes include, without limitation, a 10%, 20% or even 30% increase in throughput through enzymatic hydrolysis, saccharification and fermentation equipment; a 10%, 20% or even 30% reduction in enzyme dosage for a given glucose yield; a 10%, 15% or 20% reduction in steam consumption; and a 10%, 15% or 20% reduction in electrical power consumption. It is further believed that the present process provides for a net enzyme reduction (on a gram cellulase to gram cellulose basis) of about 25% at a 80% glucose yield; a net enzyme reduction of about 35% at a 85% glucose yield; and a net reduction of about 85% at a 90% glucose yield. In some aspects, the enzyme dosage required to achieve a given glucose yield based on cellulose content of the extracted dewatered lignocellulosic biomass is at least about 10% less, at least about 20% less or at least about 30% less than the enzyme dosage required a similar glucose yield for dewatered lignocellulosic biomass prepared by a comparable process, differing with respect to the absence of the extracting and dewatering steps. In some other aspects, the enzyme dosage required to achieve a given glucose yield based on cellulose content of the extracted dewatered lignocellulosic biomass as compared to the enzyme dosage required a similar glucose yield for dewatered lignocellulosic biomass prepared by a comparable process, differing with respect to the absence of the extracting and dewatering steps, is reduced by about 25% at a 80% glucose yield, is reduced by about 35% at a 85% glucose yield, or is reduced by about 85% at a 90% glucose yield.

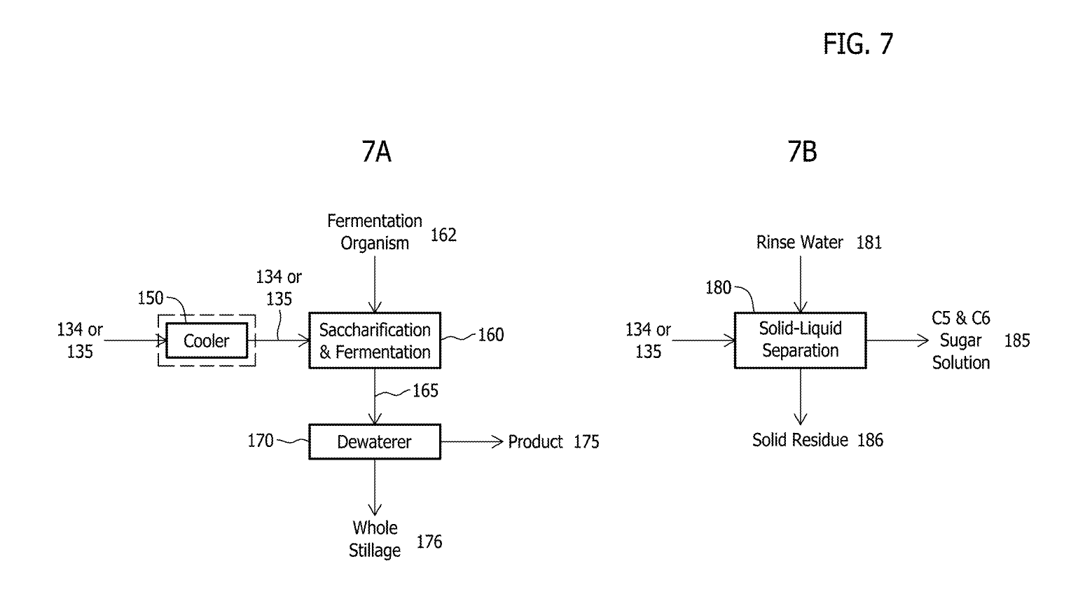

In some aspects of the disclosure, the improved cooling efficiency afforded by the process of the present disclosure provides for enzymatic hydrolyzates having a temperature of less than about 50° C. Such low temperatures eliminate the need for a cooler, such as optional cooler 150 depicted in FIG. 7a, that would otherwise be required to reduce the enzymatic hydrolyzate temperature to from about 20° C. to about 45° C., from about 25° C. to about 40° C., or from about 28° C. to about 32° C. before contact with fermentative yeast. Elimination of such a cooler provides for distinct process advantages because high solids enzymatic hydrolyzates are viscous and difficult to cool efficiently, such as with a tube and shell heat exchanger or plate heat exchanger, due to poor flowability, a tendency to foul process equipment, and a tendency to plug in process equipment.

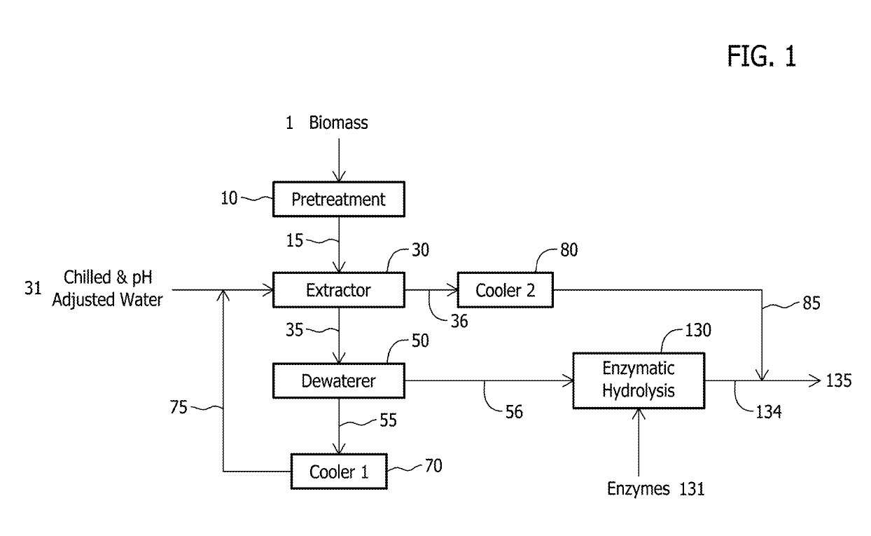

Various non-limiting aspects of the present disclosure are depicted in FIGS. 1 to 10.

FIG. 1 depicts a first aspect of the present disclosure wherein lignocellulosic biomass 1 is processed by a pretreatment step 10 comprising impregnation and steam explosion to form pretreated lignocellulosic biomass hydrolyzate 15 comprising soluble components including xylose monosaccharide. The pretreated lignocellulosic biomass hydrolyzate 15 is processed in extractor 30 by contact with chilled and pH adjusted water 31 and a chilled lean liquor stream 75, to form an adjusted extracted lignocellulosic biomass slurry 35 and a rich liquor stream 36 that is enriched in xylose as compared to the adjusted extracted lignocellulosic biomass slurry 35. In any of the various aspects of the present disclosure, the temperature and pH of the combination of the chilled and pH adjusted water 31 and the chilled lean liquor 75, and the weight ratio thereof to the pretreated lignocellulosic biomass hydrolyzate 15, are selected to yield an adjusted extracted lignocellulosic biomass slurry 35 having a total insoluble solids content less than 25 percent by weight, a pH of from about 4 to about 6, and a temperature of from about 30° C. to about 70° C. The adjusted extracted lignocellulosic biomass slurry 35 is processed in dewaterer 50 to form a dewatered lignocellulosic biomass 56 comprising at least 25 wt. % total insoluble solids and a lean liquor stream 55. The lean liquor stream 55 is passed through cooler 1 70 to from the chilled lean liquor stream 75 for recycle to the extractor 30. In some optional aspects of the disclosure, not depicted in FIG. 1, at least a portion of the lean liquor stream may be purged from the process, or may be processed to remove certain impurities (such as enzymatic and/or fermentation inhibitors) to generate a purified lean liquor stream that may be recycled at one or more points in the process. The rich liquor stream 36 is passed through cooler 2 80 to form a chilled rich liquor stream 85. The dewatered lignocellulosic biomass 56 is contacted with a source of enzymes comprising cellulase 131 in an enzymatic hydrolysis step 130 to form a lignocellulosic biomass hydrolyzate 134 comprising an admixture of hexose (“C6”) and pentose (“C5”) monosaccharides. The lignocellulosic biomass hydrolyzate 134 is combined with the chilled rich liquor stream 85 to form monosaccharide rich stream 135. In some aspects of the disclosure, at least a portion of the chilled rich liquor stream 85 may be purified to remove and/or inactivate inhibitors and be added to the enzymatic hydrolysis step 130. The lignocellulosic biomass hydrolyzate 134 is combined with the chilled rich liquor stream 85 to form monosaccharide rich stream 135. As disclosed elsewhere herein, monosaccharide rich stream 135 may be processed to form a C5 and C6 sugar solution or may be contacted with a source of fermentation organisms to form a fermentation product. In some aspects of the disclosure, the lignocellulosic biomass 1 is impregnated with acid prior to steam explosion and the chilled and pH adjusted water 31 is of alkaline pH. In some other aspects of the disclosure, the lignocellulosic biomass 1 is impregnated with a base prior to steam explosion and the chilled and pH adjusted water 31 is of acidic pH.

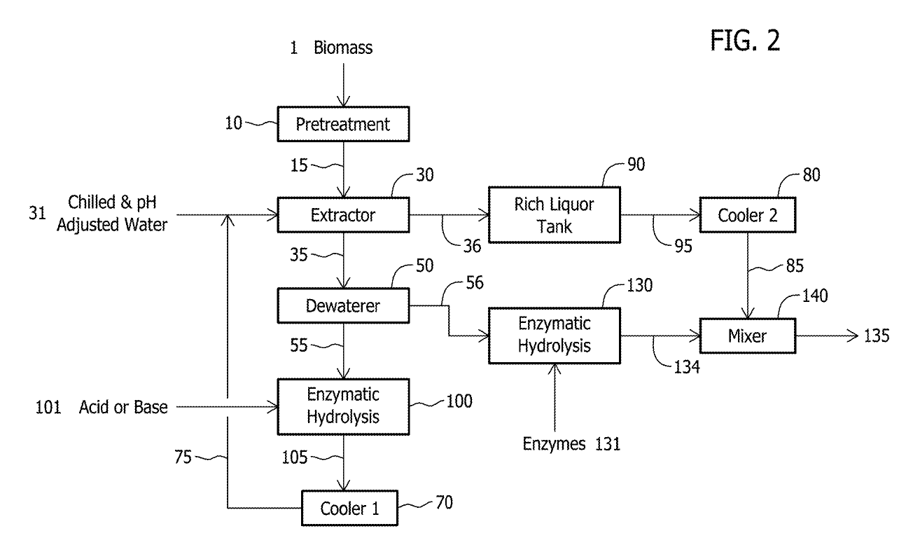

FIG. 2 depicts a second aspect of the present disclosure wherein lignocellulosic biomass 1 is processed by a pretreatment step 10 comprising impregnation with an aqueous stream (e.g., acid impregnation) and steam explosion to form pretreated lignocellulosic biomass hydrolyzate 15 comprising soluble components including xylose monosaccharide. The pretreated lignocellulosic biomass hydrolyzate 15 is processed in extractor 30 by contact with chilled and pH adjusted water 31 and a pH adusted chilled lean liquor stream 75, to form an adjusted extracted lignocellulosic biomass slurry 35 and a rich liquor stream 36 that is enriched in xylose as compared to the adjusted extracted lignocellulosic biomass slurry 35. In any of the various aspects of the present disclosure, the temperature and pH of the combination of the chilled and pH adjusted water 31 and the chilled lean liquor 75, and the weight ratio thereof to the pretreated lignocellulosic biomass hydrolyzate 15, are selected to yield an adjusted extracted lignocellulosic biomass slurry 35 having a total insoluble solids content of less than 25 wt. %, a pH of from about 4 to about 6, and a temperature of from about 30° C. to about 70° C. The adjusted extracted lignocellulosic biomass slurry 35 is processed in dewaterer 50 to form a dewatered lignocellulosic biomass 56 comprising at least 25 wt. % total insoluble solids and the lean liquor stream 55. The lean liquor stream 55 is forwarded to lean liquor tank 100 where it is combined with an acid or a base 101 to form a pH adjusted lean liquor stream 105 that is passed through cooler 1 70 to from the chilled pH adjusted lean liquor stream 75 for recycle to the extractor 30. In some optional aspects of the disclosure, not depicted in FIG. 2, at least a portion of the lean liquor stream 55, the pH adjusted lean liquor stream 105 or the chilled pH adjusted lean liquor stream 75 may be purged from the process. In some further aspects, not depicted in FIG. 2, at least a portion of any of lean liquor streams 55, 75 and/or 105 may be purified prior to recycle to extractor 30 by removal of enzymatic and/or fermentation inhibitors prior, such as by contact with an ion exchange resin or activated charcoal. The rich liquor stream 36 is forwarded to a rich liquor tank 90. Not depicted in FIG. 2, the rich liquor stream may be optionally processed in rich liquor tank 90 such as, for instance and without limitation, (i) pH adjustment by addition of an acid or a base to produce a pH adjusted rich liquor 95, (ii) removal of suspended solids such as by recycle through a filter or centrifuge to produce clarified rich liquor 95 and a solids stream for recycle to pretreatment 10 or enzymatic hydrolysis 130 and/or (iii) purification for removal of inhibitors such as by contact with an ion exchange resin or activated charcoal to produce a purified rich liquor 95. In any of the various aspects of the disclosure, the rich liquor 95 is passed through cooler 2 80 to form a chilled rich liquor stream 85. The dewatered lignocellulosic biomass 56 is contacted with a source of enzymes comprising cellulase and hemicellulase (e.g., xylanase) 131 in an enzymatic hydrolysis step 130 to form a lignocellulosic biomass hydrolyzate 134 comprising an admixture of hexose (“C6”) and pentose (“C5”) monosaccharides. The lignocellulosic biomass hydrolyzate 134 is combined with the chilled rich liquor stream 85 in mixer 140 to form monosaccharide rich stream 135. In some aspects of the disclosure, at least a portion of the chilled rich liquor stream 85 may be purified to remove and/or inactivate inhibitors and be added to the enzymatic hydrolysis step 130. As disclosed elsewhere herein, monosaccharide rich stream 135 may be processed to form a C5 and C6 sugar solution or may be contacted with a source of fermentation organisms to form a fermentation product. In some aspects of the disclosure, the lignocellulosic biomass 1 is impregnated with acid prior to steam explosion, the chilled and pH adjusted water 31 is of alkaline pH, and stream 101 is a base. In some other aspects of the disclosure, the lignocellulosic biomass 1 is impregnated with a base prior to steam explosion and the chilled and pH adjusted water 31 is of acidic pH, and stream 101 is an acid.

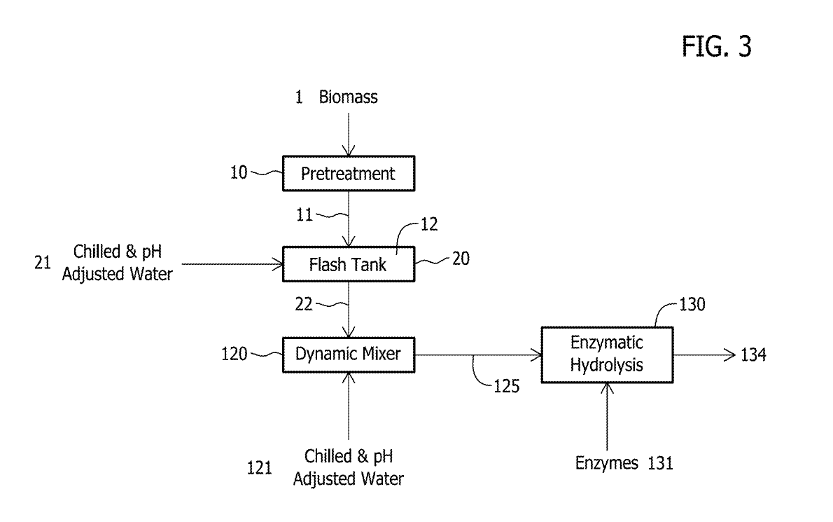

FIG. 3 depicts a third aspect of the present disclosure wherein lignocellulosic biomass 1 is processed by a pretreatment step 10 comprising impregnation (e.g., acid impregnation) and contact with steam to form lignocellulosic biomass 11 having elevated temperature and pressure. The pressure of the lignocellulosic biomass 11 is reduced in flash tank 20 to form pretreated lignocellulosic biomass 12 having elevated temperature. In some alternative aspects of the disclosure, (i) flash tank 20 is integral to, or is the same vessel as, the pressurization vessel of pretreatment step 10 or (ii) pressurized lignocellulosic biomass is transferred from the pressurization vessel of pretreatment 10 to flash tank 20. Pretreated biomass 12 is contacted with chilled and pH adjusted water 21 in the flash tank 20 to form cooled and pH adjusted pretreated lignocellulosic biomass 22. Pretreated lignocellulosic biomass 22 is contacted with additional chilled and pH adjusted water 121 in dynamic mixer 120 to form adjusted pretreated lignocellulosic biomass 125. The temperature and pH of the chilled and pH adjusted water 21 and the weight ratio thereof to the pretreated biomass 12 and the temperature and pH of the chilled and pH adjusted water 121 and the weight ratio thereof to the pretreated lignocellulosic biomass 22 are selected to yield an adjusted pretreated lignocellulosic biomass 125 having a pH of from about 4 to about 6, a temperature of from about 30° C. to about 70° C., and a total insoluble solids content of at least 22 wt. %. The adjusted pretreated lignocellulosic biomass 125 is contacted with a source of enzymes comprising cellulase 131 in an enzymatic hydrolysis step 130 to form a lignocellulosic biomass hydrolyzate 134 comprising an admixture of hexose (“C6”) and pentose (“C5”) monosaccharides. As disclosed elsewhere herein, monosaccharide rich stream 134 may be processed to form a C5 and C6 sugar solution or may be contacted with a source of fermentation organisms to form a fermentation product. In some aspects of the disclosure, the lignocellulosic biomass 1 is impregnated with acid prior to steam explosion and the chilled and pH adjusted water 21 and 121 are of alkaline pH. In some other aspects of the disclosure, the lignocellulosic biomass 1 is impregnated with a base prior to steam explosion and the chilled and pH adjusted water 21 and 121 are of acidic pH.

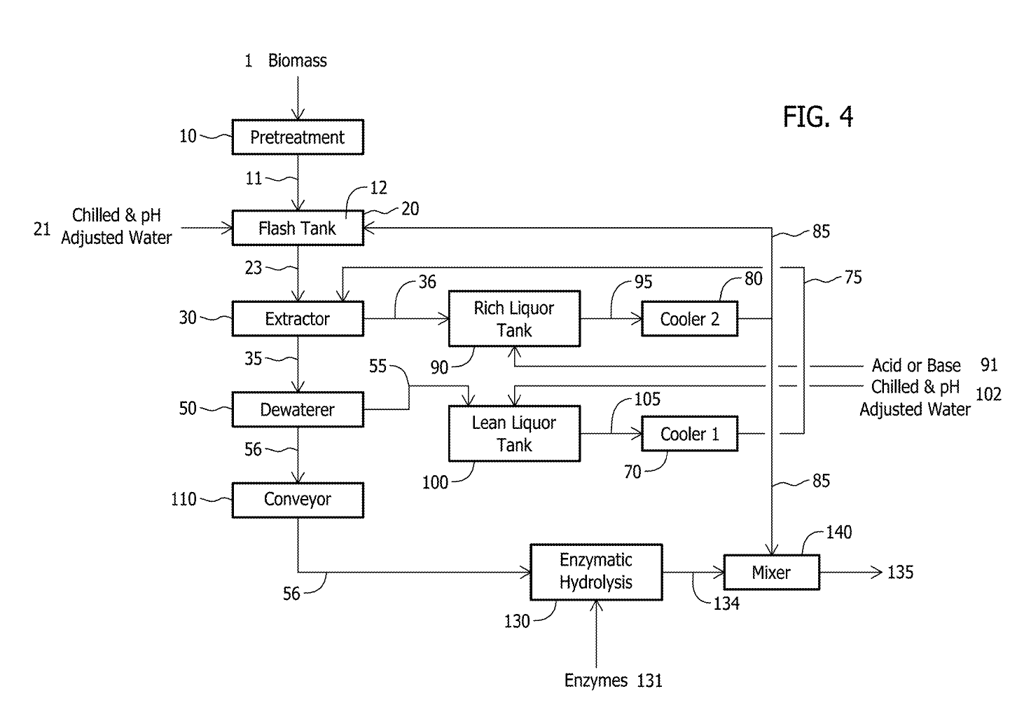

FIG. 4 depicts a fourth aspect of the present disclosure wherein lignocellulosic biomass 1 is processed by a pretreatment step 10 comprising impregnation (e.g., acid impregnation) and contact with steam to form lignocellulosic biomass 11 having elevated temperature and pressure. The pressure of the lignocellulosic biomass 11 is reduced in flash tank 20 to form pretreated lignocellulosic biomass 12 having elevated temperature. In some alternative aspects of the disclosure, (i) flash tank 20 is integral to, or is the same vessel as, the pressurization vessel of pretreatment step 10 or (ii) pressurized lignocellulosic biomass is transferred from the pressurization vessel of pretreatment 10 to flash tank 20. Pretreated biomass 12 is contacted with chilled and pH adjusted water 21 and at least a portion of a adjusted rich liquor stream 85 in the flash tank 20 to form cooled and pH adjusted pretreated lignocellulosic biomass 23. The cooled and pH adjusted pretreated lignocellulosic biomass 23 is processed in extractor 30 by contact with chilled and pH adjusted lean liquor stream 75 to form an adjusted extracted lignocellulosic biomass slurry 35 and a rich liquor stream 36 that is enriched in xylose as compared to the adjusted extracted lignocellulosic biomass slurry 35. In any of the various aspects of the present disclosure, the temperature and pH of the chilled and pH adjusted water 21 and the weight ratio thereof to the pretreated biomass 12 and the temperature and pH of the chilled and pH adjusted lean liquor stream 75 and the weight ratio thereof to the pretreated lignocellulosic biomass 23 are selected to yield an extracted pretreated lignocellulosic biomass 35 having a pH of from about 4 to about 6, a temperature of from about 30° C. to about 70° C., and a total insoluble solids content of less than 25 wt. %. The adjusted extracted lignocellulosic biomass slurry 35 is processed in dewaterer 50 to form a dewatered lignocellulosic biomass 56 comprising at least 25 wt. % total insoluble solids and a lean liquor stream 55. The lean liquor stream 55 is forwarded to lean liquor tank 100 where it is combined with chilled and pH adjusted water 102 to form a pH adjusted lean liquor stream 105 that is passed through cooler 1 70 to from the chilled pH adjusted lean liquor stream 75 for recycle to the extractor 30. In some optional aspects of the disclosure, not depicted in FIG. 4, at least a portion of the lean liquor stream 55, the pH adjusted lean liquor stream 105 or the chilled pH adjusted lean liquor stream 75 may be purged from the process. In some further aspects, not depicted in FIG. 4, (i) at least a portion of any of lean liquor streams 55, 75 and/or 105 may be processed to remove suspended solids (e.g., fines and sand) prior to recycle to extractor 30. The rich liquor stream 36 is forwarded to a rich liquor tank 90. The pH of the rich liquor stream 36 is adjusted in the rich liquor tank 90 by the addition of an acid or a base 91. In any of the various aspects of the disclosure, the adjusted rich liquor 95 is passed through cooler 2 80 to form a chilled pH adjusted rich liquor stream 85. Not depicted in FIG. 4, at least a portion of any of rich liquor stream 36, 95 or 85 may be purified prior to recycle by (i) removal of enzymatic and/or fermentation inhibitors prior, such as by contact with an ion exchange resin or activated charcoal and/or (ii) removal of suspended solids including impurities such as by recycle through a filter or centrifuge. The chilled and pH adjusted rich liquor stream 85 may be recycled to the flash tank 20, may be forwarded to mixer 140 for combination with monosaccharide rich stream 134 or may be split between recycle to flash tank 20 and mixer 140. The dewatered lignocellulosic biomass 56 is transferred from the dewaterer 50 to the enzymatic hydrolysis step 130 by conveyor 110. The dewatered lignocellulosic biomass 56 is contacted with a source of enzymes comprising cellulase and hemicellulase (e.g., xylanase) 131 in the enzymatic hydrolysis step 130 to form a lignocellulosic biomass hydrolyzate 134 comprising an admixture of hexose (“C6”) and pentose (“C5”) monosaccharides. The lignocellulosic biomass hydrolyzate 134 is combined with at least a portion of the chilled and pH adjusted rich liquor stream 85 in mixer 140 to form monosaccharide rich stream 135. In some aspects of the disclosure, at least a portion of the chilled and pH adjusted rich liquor stream 85 (such as, for example a purified rich liquor stream) may be added to the enzymatic hydrolysis step 130. As disclosed elsewhere herein, monosaccharide rich stream 135 may be processed to form a C5 and C6 sugar solution or may be contacted with a source of fermentation organisms to form a fermentation product. In some aspects of the disclosure, the lignocellulosic biomass 1 is impregnated with acid prior to steam explosion, the chilled and pH adjusted water 21 and 102 are of alkaline pH, and stream 91 is a base. In some other aspects of the disclosure, the lignocellulosic biomass 1 is impregnated with a base prior to steam explosion, the chilled and pH adjusted water 21 and 102 are of acidic pH, and stream 91 is an acid.

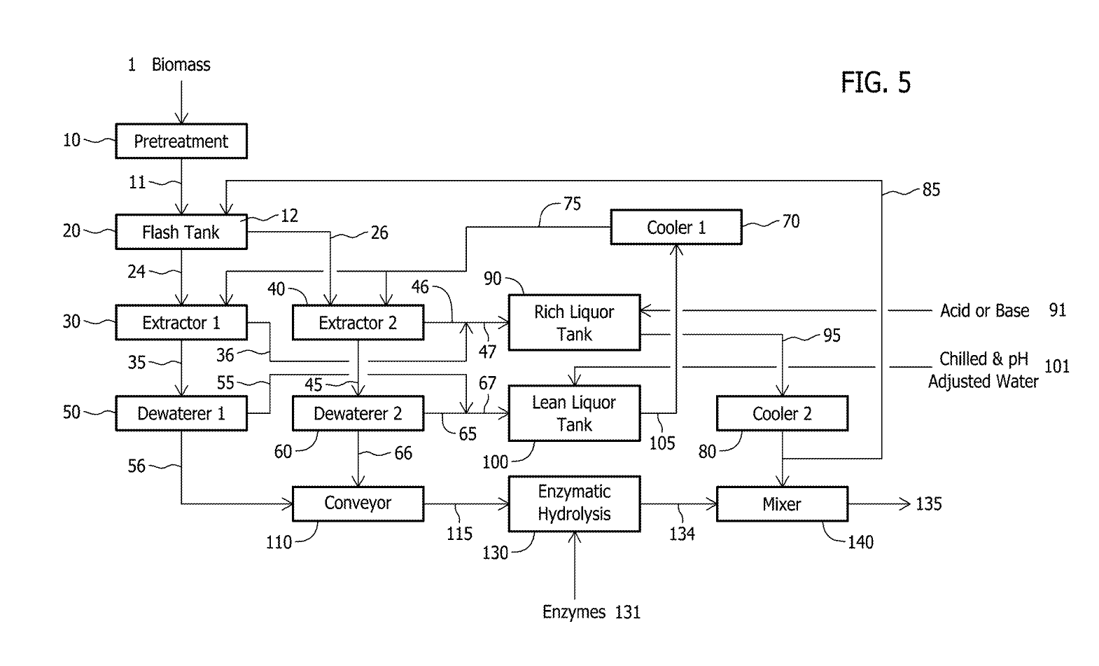

FIG. 5 depicts a fifth aspect of the present disclosure wherein lignocellulosic biomass 1 is processed by a pretreatment step 10 comprising impregnation (e.g., acid impregnation) and contact with steam to form lignocellulosic biomass 11 having elevated temperature and pressure. The pressure of the lignocellulosic biomass 11 is reduced in flash tank 20 to form pretreated lignocellulosic biomass 12 having elevated temperature. In some alternative aspects of the disclosure, (i) flash tank 20 is integral to, or is the same vessel as, the pressurization vessel of pretreatment step 10 or (ii) pressurized lignocellulosic biomass is transferred from the pressurization vessel of pretreatment 10 to flash tank 20. Pretreated biomass 12 is contacted with at least a portion of a chilled and pH adjusted rich liquor stream 85 in the flash tank to form cooled and pH adjusted pretreated lignocellulosic biomass. A first cooled and pH adjusted pretreated lignocellulosic biomass 24 stream is processed in extractor 30 and a second pH adjusted pretreated lignocellulosic biomass stream 26 stream is processed in extractor 40. In extractor 30, the pretreated lignocellulosic biomass 24 is processed by contact with chilled and pH adjusted lean liquor stream 75 to form an adjusted extracted lignocellulosic biomass slurry 35 and a rich liquor stream 36 that is enriched in xylose as compared to the adjusted extracted lignocellulosic biomass slurry 35. In any of the various aspects of the present disclosure, the temperature and pH of the chilled and pH adjusted rich liquor stream 85 and the weight ratio thereof to the pretreated biomass 12 and the temperature and pH of the chilled and pH adjusted lean liquor stream 75 and the weight ratio thereof of the pretreated biomass 24 are selected to yield an extracted pretreated lignocellulosic biomass 35 having a pH of from about 4 to about 6, a temperature of from about 30° C. to about 70° C., and a total insoluble solids content of less than 25 wt. %. The adjusted extracted lignocellulosic biomass slurry 35 is processed in dewaterer 50 to form a dewatered lignocellulosic biomass 56 comprising at least 25 wt. % total insoluble solids and a lean liquor stream 55. The lean liquor stream 55 is combined with lean liquor stream 65 from dewaterer 60 to form combined lean liquor stream 67 that is forwarded to lean liquor tank 100 where the combined streams are admixed with chilled and pH adjusted water 101 to form a pH adjusted lean liquor stream 105 that is passed through cooler 1 70 to form the adjusted chilled lean liquor stream 75 for recycle to the extractor 30 and extractor 40. The second cooled and pH adjusted pretreated lignocellulosic biomass stream 26 stream is processed in extractor 40. In extractor 40, the pretreated biomass 26 is processed by contact with chilled and pH adjusted lean liquor stream 75 to form an adjusted extracted lignocellulosic biomass slurry 45 and a rich liquor stream 46 that is enriched in xylose as compared to the adjusted extracted lignocellulosic biomass slurry 45. The rich liquor stream 36 from extractor 30 is combined with rich liquor stream 46 to form a combined rich liquor stream 47 that is forwarded to rich liquor tank 90. The pH of the rich liquor stream 47 is adjusted in the rich liquor tank 90 by the addition of an acid or a base 91 to form the pH adjusted rich liquor stream 95 that is passed through cooler 2 80 to form the pH adjusted chilled rich liquor stream 85 for recycled to the flash tank 20 and/or for combination with lignocellulosic biomass hydrolyzate 134 in mixer 140. Not depicted in FIG. 5, at least a portion of any of the rich liquor streams 36, 46, 47, 95 or 85 may be purified prior to recycle by (i) removal of enzymatic and/or fermentation inhibitors prior, such as by contact with an ion exchange resin or activated charcoal and/or (ii) removal of suspended solids including impurities such as by recycle through a filter or centrifuge to remove precipitated impurities. Additionally, any of the rich liquor streams 36, 46, 47 may be optionally filtered or centrifuged to recover insoluble lignocellulosic material for recycle to pretreatment 10 or to enzyme hydrolysis 130. In any of the various aspects of the present disclosure, the temperature and pH of the chilled and pH rich liquor stream 85 and the weight ratio thereof to the pretreated biomass 12 and the temperature and pH of the chilled and pH adjusted lean liquor stream 75 and the weight ratio thereof of the pretreated biomass 26 are selected to yield an extracted pretreated lignocellulosic biomass 45 having a pH of from about 4 to about 6, a temperature of from about 30° C. to about 70° C., and a total insoluble solids content of less than 25 wt. %. The adjusted extracted lignocellulosic biomass slurry 45 is processed in dewaterer 60 to form a dewatered lignocellulosic biomass 66 comprising at least 25 wt. % total insoluble solids and the lean liquor stream 65. In some optional aspects of the disclosure, not depicted in FIG. 5, at least a portion of any one of the lean liquor stream 55, 65 or 67, the pH adjusted lean liquor stream 105 or the chilled pH adjusted lean liquor stream 75 may be purged from the process. In some further aspects, not depicted in FIG. 5, at least a portion of any of lean liquor streams 55, 65, 67, 75 or 105 may be purified prior to recycle to extractor 30 or 40 by removal of enzymatic and/or fermentation inhibitors, such as by contact with an ion exchange resin or activated charcoal and/or (ii) at least a portion of any of alkaline lean liquor streams 75 and/or 105 may be filtered or centrifuged prior to recycle to extractor 30 to remove insoluble solid impurities. The dewatered lignocellulosic biomass 56 and the dewatered lignocellulosic biomass 66 are transferred by conveyor 110 as combined dewatered lignocellulosic biomass 115 to the enzymatic hydrolysis step 130. The dewatered lignocellulosic biomass 115 is contacted with a source of enzymes comprising cellulase and hemicellulase (e.g., xylanase) 131 in the enzymatic hydrolysis step 130 to form a lignocellulosic biomass hydrolyzate 134 comprising an admixture of hexose (“C6”) and pentose (“C5”) monosaccharides. The lignocellulosic biomass hydrolyzate 134 is combined with the chilled rich liquor stream 85 in mixer 140 to form monosaccharide rich stream 135. In some aspects of the disclosure, at least a portion of the chilled rich liquor stream 85 (such as, for example, a purified rich liquor stream) may be added to the enzymatic hydrolysis step 130. As disclosed elsewhere herein, monosaccharide rich stream 135 may be processed to form a C5 and C6 sugar solution or may be contacted with a source of fermentation organisms to form a fermentation product. In some aspects of the disclosure, the lignocellulosic biomass 1 is impregnated with acid prior to steam explosion, the chilled and pH adjusted water 101 is of alkaline pH, and stream 91 is a base. In some other aspects of the disclosure, the lignocellulosic biomass 1 is impregnated with a base prior to steam explosion, the chilled and pH adjusted water 101 is of acidic pH, and stream 91 is an acid.

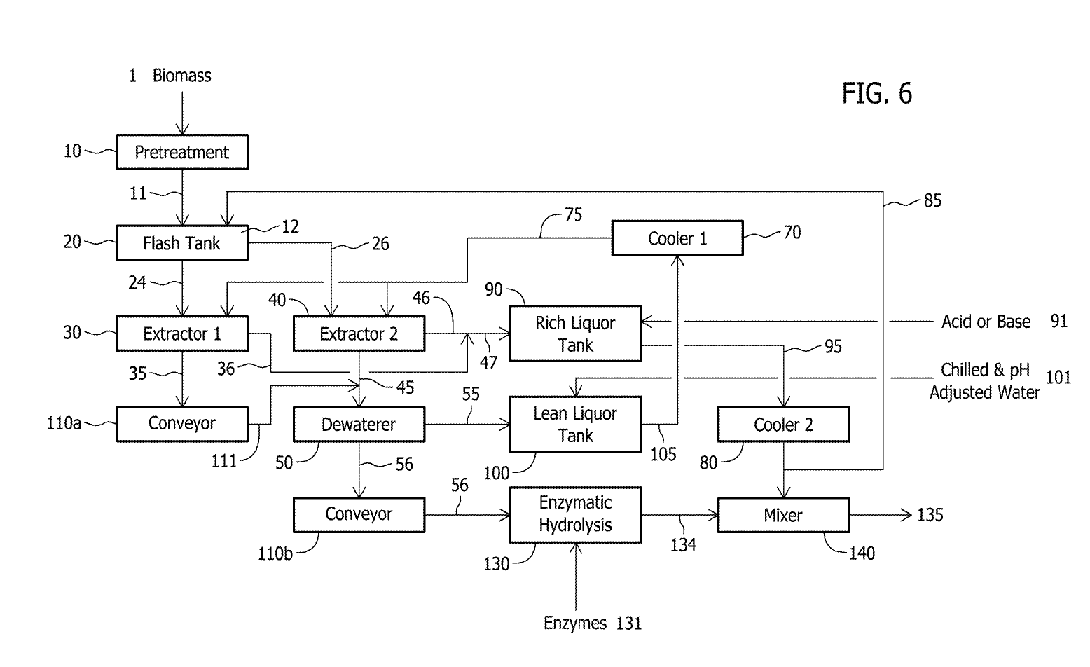

FIG. 6 depicts a sixth aspect of the present disclosure wherein lignocellulosic biomass 1 is processed by a pretreatment step 10 comprising impregnation (e.g., acid impregnation) and contact with steam to form lignocellulosic biomass 11 having elevated temperature and pressure. The pressure of the lignocellulosic biomass 11 is reduced in flash tank 20 to form pretreated lignocellulosic biomass 12 having elevated temperature. In some alternative aspects of the disclosure, (i) flash tank 20 is integral to, or is the same vessel as, the pressurization vessel of pretreatment step 10 or (ii) pressurized lignocellulosic biomass is transferred from the pressurization vessel of pretreatment 10 to flash tank 20. Pretreated biomass 12 is contacted with chilled and pH adjusted rich liquor stream 85 in the flash tank to form cooled and pH adjusted pretreated lignocellulosic biomass. A first cooled and pH adjusted pretreated lignocellulosic biomass stream 24 stream is processed in extractor 30 and a second pH adjusted pretreated lignocellulosic biomass stream 26 stream is processed in extractor 40. In extractor 30, the pretreated biomass 24 is processed by contact with chilled and pH adjusted lean liquor stream 75 to form an adjusted extracted lignocellulosic biomass slurry 35 and a rich liquor stream 36 that is enriched in xylose as compared to the adjusted extracted lignocellulosic biomass slurry 35. In any of the various aspects of the present disclosure, the temperature and pH of the chilled and pH rich liquor stream 85 and the weight ratio thereof to the pretreated biomass 12 and the temperature and pH of the chilled and pH adjusted lean liquor stream 75 and the weight ratio thereof of the pretreated biomass 24 are selected to yield an extracted pretreated lignocellulosic biomass 35 having a pH of from about 4 to about 6, a temperature of from about 30° C. to about 70° C., and a total insoluble solids content of less than 25 wt. %. The adjusted extracted lignocellulosic biomass slurry 35 is transferred by conveyor 110a to dewaterer 50 where it is combined with adjusted extracted lignocellulosic biomass slurry 45 and dewatered to form a dewatered lignocellulosic biomass 56 comprising at least 25 wt. % total insoluble solids and the lean liquor stream 55. The lean liquor stream 55 is forwarded to lean liquor tank 100 where it is admixed with chilled and pH adjusted water 101 to form a pH adjusted lean liquor stream 105 that is passed through cooler 1 70 to form the adjusted chilled lean liquor stream 75 for recycle to the extractor 30 and extractor 40. A second cooled and pH adjusted pretreated lignocellulosic biomass stream 26 stream is processed in extractor 40. In extractor 40, the pretreated biomass 26 is processed by contact with chilled and pH adjusted lean liquor stream 75 to form an adjusted extracted lignocellulosic biomass slurry 45 and a rich liquor stream 46 that is enriched in xylose as compared to the adjusted extracted lignocellulosic biomass slurry 45. The rich liquor stream 36 is combined with rich liquor stream 46 to form rich liquor stream 47 that is forwarded to rich liquor tank 90 where the combined streams are admixed with an acid or a base 91 to form a pH adjusted rich liquor stream 95 that is passed through cooler 2 80 to form the adjusted chilled rich liquor stream 85 for recycled to the flash tank 20 and for combination with lignocellulosic biomass hydrolyzate 134 in mixer 140. Not depicted in FIG. 6, any of the rich liquor streams 36, 46, 47, 95 or 85 may be purified prior to recycle by (i) removal of enzymatic and/or fermentation inhibitors prior, such as by contact with an ion exchange resin or activated charcoal and/or (ii) removal of suspended solids including impurities such as by recycle through a filter or centrifuge to remove precipitated impurities. Additionally, any of the rich liquor streams 36, 46, 47 may be optionally filtered or centrifuged to recover insoluble lignocellulosic material for recycle to pretreatment 10 or to enzyme hydrolysis 130. In any of the various aspects of the present disclosure, the temperature and pH of the chilled and pH rich liquor stream 85 and the weight ratio thereof to the pretreated biomass 12 and the temperature and pH of the chilled and pH adjusted lean liquor stream 75 and the weight ratio thereof of the pretreated biomass 26 are selected to yield an extracted pretreated lignocellulosic biomass 45 having a pH of from about 4 to about 6, a temperature of from about 30° C. to about 70° C., and a total insoluble solids content of less than 25 wt. %. In some optional aspects of the disclosure, not depicted in FIG. 6, at least a portion of any one of the lean liquor stream 55, 65 or 67, the pH adjusted lean liquor stream 105 or the chilled pH adjusted lean liquor stream 75 may be purged from the process. In some further aspects, not depicted in FIG. 6, at least a portion of any of lean liquor streams 55, 65, 67, 75 or 105 may be purified prior to recycle to extractor 30 or 40 by removal of enzymatic and/or fermentation inhibitors, such as by contact with an ion exchange resin or activated charcoal and/or (ii) at least a portion of any of alkaline lean liquor streams 75 and/or 105 may be filtered or centrifuged prior to recycle to extractor 30 to remove insoluble solid impurities. The dewatered lignocellulosic biomass 56 is transferred by conveyor 110b to the enzymatic hydrolysis step 130. The dewatered lignocellulosic biomass 56 is contacted with a source of enzymes comprising cellulase 131 in the enzymatic hydrolysis step 130 to form a lignocellulosic biomass hydrolyzate 134 comprising an admixture of hexose (“C6”) and pentose (“C5”) monosaccharides. The lignocellulosic biomass hydrolyzate 134 is combined with at least a portion of the chilled rich liquor stream 85 in mixer 140 to form monosaccharide rich stream 135. In some aspects of the disclosure, at least a portion of the chilled rich liquor stream 85 (such as, for example, a purified rich liquor stream) may be added to the enzymatic hydrolysis step 130. As disclosed elsewhere herein, monosaccharide rich stream 135 may be processed to form a C5 and C6 sugar solution or may be contacted with fermentation organisms to form a fermentation product. In some aspects of the disclosure, the lignocellulosic biomass 1 is impregnated with acid prior to steam explosion, the chilled and pH adjusted water 101 is of alkaline pH, and stream 91 is a base. In some other aspects of the disclosure, the lignocellulosic biomass 1 is impregnated with a base prior to steam explosion, the chilled and pH adjusted water 101 is of acidic pH, and stream 91 is an acid.

FIG. 7 depicts two aspects of the disclosure directed to processing lignocellulosic biomass hydrolyzate 134 and monosaccharide rich stream 135. In a first aspect, depicted in FIG. 7a, lignocellulosic biomass hydrolyzate 134 or monosaccharide rich stream 135 are typically of a temperature sufficiently low for fermentation without additional temperature reduction, and may be contacted with a source of fermentation organisms 162 in a fermentation step 160 and held for a time period sufficient to form a fermentation mixture 165 comprising a recoverable amount of a fermentation product 175. In some optional aspects, lignocellulosic biomass hydrolyzate 134 or monosaccharide rich stream 135 may be passed through a cooler 150 to further reduce the temperature prior to contact with the source of fermentation organisms 162 to form the fermentation mixture 165. The saccharification and fermentation steps may suitably be done separately or simultaneously. In some aspects, depicted in FIG. 7a, the fermentation mixture 165 may be processed by distillation 170 to form the fermentation product 175 and whole stillage 176. In a second aspect, depicted in FIG. 7b, lignocellulosic biomass hydrolyzate 134 or monosaccharide rich stream 135 are fractionated by solid-liquid separation 180 to form a C5 & C6 sugar solution 185 and solid residue 186 wherein the solid residue may be washed with rinse water 181 to generate additional C5 & C6 sugar solution 185.

Lignocellulosic biomass material suitable for the practice of the present disclosure includes, without limitation, corn stover, corn cobs, corn fibers, straw, banana plantation waste, rice straw, rice hull, oat straw, oat hull, corn fiber, cotton stalks, wheat straw, sugar cane bagasse, sugar cane straw, sorghum residues, sugar processing residues, barley straw, cereal straw, wheat straw, canola straw, soybean stover, woody biomass, switchgrass, cordgrass, ryegrass, miscanthus, alfalfa, residential waste, food waste, paper waste and boards, and papermill sludge. Lignocellulosic biomass composition varies with the source, but typically comprises from about 25 wt. % to about 55 wt. % or from about 30 wt. % to about 40 wt. % cellulose, from about 15 wt. % to about 35 wt. % or from about 20 wt. % to about 30 wt. % hemicellulose, from about 10 wt. % to about 30 wt. % or from about 15 wt. % to about 25 wt. % lignin, and from about 2 wt. % to about 10 wt. % or from about 3 wt. % to about 6 wt. % ash, where woody biomass generally comprises less than about 0.5 wt. % ash. Ash generally comprises various inorganic elements such as silica, calcium, magnesium, sodium, potassium, phosphorus, aluminum, and combinations thereof.

Prior to pretreatment, lignocellulosic biomass feedstock may optionally be subjected to particle size reduction to reduce the particle size to a generally homogeneous range. The lignocellulosic biomass feedstock may further optionally be cleaned to remove impurities and contaminants. The feedstock may be suitably comminuted in a grinder, hammer mill or other suitable comminuting device known in the art. Generally, comminuted feedstock predominantly contains particles having a size in their largest dimension of less than about 6 cm, less than about 5 cm, less than about 4 cm, or less than about 2.5 cm. Typically, such feedstock contains particles of a size from about 0.01 cm to about 6 cm, from about 0.1 cm to about 5 cm, from about 0.5 cm to about 4 cm.

Lignocellulosic feedstock or comminuted lignocellulosic feedstock may optionally be processed to remove various impurities and contaminants (e.g., rock, dirt, sand, and other materials) and feedstock particles of undesired size, such a fine particles. Cleaning of the milled feedstock proceeds generally by methods known in the art including, for example, by a process comprising passing the feedstock over a suitable screen that separates desired and undesired particles. Typically, desired and undesired particles are separated by vibration and/or shaking of the screen. Contaminants (e.g., ferrous contaminants) and oversized particles and fines may also be removed by magnetic separation. Contaminants may also be removed from the feedstock by density separation by contact with a suitable flow of air (i.e., air classification) and/or by contact with an aqueous washing medium (e.g., water). Fine particles are known to be enriched in ash (such as up to about 40 wt. % ash) as compared to the remainder of the feedstock. Ash components may interfere with acid impregnation for the purpose of preparation of the feedstock for enzymatic hydrolysis to produce fermentable sugars and may increase acid consumption required to reach a desired pH due to acid neutralizing capacity. Ash removal from the feedstock may be conducted by, for example, washing of the acid-impregnated feedstock as detailed elsewhere herein, or by classification of particulate material. Suitable classification methods include air classifying, screen separation, filtration, and sedimentation techniques or by means of a cyclone separator. In screen separation, the screen typically has openings of a size of from about U.S. Sieve No. 100 (150 μm) to about U.S. Sieve No. 20 (840 μm) or from about U.S. Sieve No. 80 (175 μm) to about U.S. Sieve No. 60 (250 μm). For example, typically the screen separation system comprises a screen having openings of a size of about U.S. Sieve No. 20 (840 μm), about U.S. Sieve No. 60 (250 μm), about U.S. Sieve No. 80 (175 μm), or about U.S. Sieve No. 100 (150 μm) wherein the ash rich fraction is the undersize (fines) fraction.

In any of the various aspects of the disclosure, the lignocellulosic biomass feedstock for pretreatment typically is impregnated with an aqueous medium to achieve a moisture content of from about 20 wt. % to about 80 wt. %, from about 30 wt. % to about 70 wt. %, from about 30 wt. % to about 60 wt. %, from about 35 wt. % to about 60 wt. %, or from about 40 wt. % to about 60 wt. %. In some aspects, the aqueous medium is a dilute acid and the impregnated lignocellulosic biomass feedstock comprises acid in a final dosage (i.e., the acid loading in the dewatered acid-impregnated biomass entering the pretreatment reactor vessel) of from about 0.01 to about 0.05 kg acid per kg lignocellulosic biomass, or from about 0.02 to about 0.04 kg acid per kg lignocellulosic biomass, on a solids basis. Alternatively stated, the pH of acid impregnated biomass is about 1, 2, 3, 4 or 5, and ranges thereof, such as from about 1 to about 5, less than 4, from about 1 to about 4, or from about 1 to about 2.5. Mineral acids (e.g., sulfuric acid, nitric acid, phosphoric acid, and hydrochloric acid) or organic acids may be used, and mineral acids are generally preferred. In some other aspects, the aqueous medium is a dilute base and the impregnated lignocellulosic biomass feedstock comprises a base in a final dosage of from about 0.01 to about 1 kg per kg of lignocellulosic biomass, from about 0.1 to about 1 kg per kg of lignocellulosic biomass or from about 0.5 to about 1 kg per kg of lignocellulosic biomass, on a solids basis. Alternatively stated, the pH of ammonia impregnated biomass is about 8, 9, 10, 11, or 12, and ranges thereof, such as from about 8 to about 12, from about 9 to about 11. Suitable bases include sodium hydroxide, potassium hydroxide, calcium oxide, ammonia and ammonium hydroxide. In any of the various impregnation aspects, the temperature of the impregnated lignocellulosic biomass is about 30° C., about 40° C., about 50° C., about 60° C., about 70° C. or about 80° C., and ranges thereof, such as from about 30° C. to about 80° C. or from about 40° C. to about 60° C.

Lignocellulosic biomass impregnation may be done by any suitable means known in the art. In one method, particulate lignocellulosic biomass feedstock is sprayed with water (optionally comprising acid or base) with mixing in a high shear mixer, such as a ribbon blender or a pug mill. The impregnated material is typically held for a sufficient period of time prior to steam pretreatment to allow for moisture and temperature equilibration, such as about 5 minutes, 15 minutes, 30 minutes, 45 minutes or an hour. In such spraying/mixing aspects, the liquid-to-dry biomass weight ratio may be at least about 2:1, at least about 3:1 or at least about 4:1 (e.g., from about 3:1 to 8:1). In another method, a slurry comprising particulate lignocellulosic biomass feedstock and water (optionally comprising acid or base) is formed with mixing in a suitable vessel, such as a stirred tank reactor. In such aspects, the liquid-to-dry biomass weight ratio may be at least about 10:1, at least about 15:1 or at least about 20:1 (e.g., from about 15:1 to about 30:1). Optionally, an insoluble inerts stream (e.g., sand, rocks, etc.) may be removed from slurried lignocellulosic biomass by screening or density separation (e.g., settling, cyclones, or centrifugation).

The impregnated lignocellulosic biomass may undergo a dewatering operation to reduce the moisture content of the biomass to an amount suitable for steam pretreatment. Suitable equipment for dewatering includes, for example dewatering screens, centrifuges, filters and hydro-cyclones which are typically used for slurries having a total solids content of about 5 wt. % or less; screens and drain-screws which are typically used for inlet slurries having a total solids content of about 5 wt. % to about 20 wt. %; and screw presses and plug feeders which are typically used for inlet slurries having a total solids content of about 15 wt. % to about 40 wt. %. Dewatering operations may increase the total solids content of the biomass to about 30 wt. % or more, to about 40 wt. % or more, to about 50 wt. % or more (e.g., from about 30 wt. % to about 50 wt. % or from about 30 wt. % to about 40 wt. % total solids).

After dewatering, the dewatered impregnated biomass and steam are introduced into a suitable vessel wherein the impregnated lignocellulosic biomass is contacted with steam at elevated temperature and pressure followed by rapid depressurization in a steam pretreatment step to form a lignocellulosic hydrolyzate characterized by a soluble hemicellulosic sugar component and a solid lignocellulosic component having enhanced bioavailably and accessibility to enzymes. The impregnated lignocellulosic biomass may be pressed to form a cake, or plug of treated solids for introduction into the steam treatment vessel. More particularly, the solid biomass stream may be subjected to elevated pressure and temperature conditions to break down the cellulose-hemicellulose and cellulose-hemicellulose-lignin complexes, and to partially convert hemicellulose (xylan) to xylose. The precise form and configuration of the vessel is not narrowly critical and may be selected by one skilled in the art depending on the particular circumstances (e.g., properties of the lignocellulosic biomass and operating conditions). The vessel may have any suitable shape (e.g., cylindrical) and may have a vertical or horizontal orientation. The vessel may be a batch reactor or a continuous reactor. Generally, the vessel includes an inlet for introduction of the solid biomass stream and one or more outlets for releasing treated lignocellulosic biomass and/or various components generated during the steam treatment. Once the solid biomass stream is contained in the vessel, steam is injected into the vessel to pressurize and directly heat the solid biomass stream. In any of the various steam pretreatment aspects of the disclosure, a vapor or gas stream may be continuously or periodically vented from the steam pretreatment vessel to purge noncondensable gas (e.g., air) and volatile organic compounds (“VOCs”) generated as byproducts of cellulose, hemicellulose and lingo-cellulose hydrolysis that are known to be enzymatic and/or fermentation inhibitor compounds. Such inhibitors include, for instance, acetic acid, furfural and hydroxymethylfurfural (“HMF”). In some other optional aspects of the disclosure, solid biomass stream heating can additionally be done indirectly, such as by applying steam to a vessel jacket. Typically, the solid biomass stream is maintained at a target temperature and pressure, such as by pressure control, for a time sufficient to provide suitable heating. After a period of contact time, the pressure of the solid biomass stream is reduced and/or the treated feedstock is discharged to an environment of reduced pressure, such as from about 3 psig to about 20 psig, from about 3 psig to about 15 psig to generate steam treated solid biomass stream and flash and vent steam. In some aspects of the present disclosure, after a period of pressurizing the vessel and heating the solid biomass stream, the solid biomass stream is released or transferred from the contact vessel to a receiving vessel having reduced and controlled pressure. In some other aspects of the present disclosure, after a period of pressurizing the vessel and heating the solid biowaste stream, the pressure and temperature in the vessel is reduced to an intermediate pressure and temperature and held for a period of time at those conditions, followed by pressure reduction as described herein. In any of the various aspects, the change in pressure results in rapid material expansion thereby assisting in breaking down the biomass fiber structure including, for example, the bonds between lignin and hemicellulose and/or cellulose in the cellulose-hemicellulose or cellulose-hemicellulose-lignin complex (collectively termed “cellulose complexes”), increasing the available surface area and reducing the particle size. More particularly, by physio-chemical means, steam treatment typically dissociates cellulose from hemicellulose and lignin (if present) thereby providing cellulose suitable for enzymatic hydrolysis to glucose. For example, in various aspects, at least about 60 wt. %, at least about 70 wt. %, at least about 80 wt. %, or up to 90 wt. % of the cellulose contained in the cellulose complex is dissociated therefrom. Steam treatment also typically dissociates hemicellulose from the complex, generally in the form of hemicellulose solubilized within a liquid phase of the treated lignocellulosic biomass. In various aspects, at least about 60 wt. %, at least about 70 wt. %, at least about 80 wt. %, or up to 90 wt. % of the hemicellulose contained in the lignocellulosic biomass is solubilized within a liquid phase of the treated lignocellulosic biomass. In this manner, steam treatment provides cellulose and hemicellulose suitable for enzymatic hydrolysis to monosaccharides.

In some aspects of the disclosure, a two-stage pretreatment regimen is used in a batch or continuous scheme. In a continuous scheme, the lignocellulosic feedstock is continuously exposed to steam within a contact zone for the first stage of pretreatment while steam treated feedstock provided by the first stage of pretreatment is continuously subjected to the depressurization within a depressurization zone wherein a volatilized fraction is released from the depressurization zone. The two-stage process may utilize a single or multiple vessels. That is, in various embodiments the contact zone for the first stage of pretreatment and the depressurization zone for the second stage of pretreatment are contained in a single vessel. In various other embodiments, the contact zone and depressurization zone are contained in separate vessels. For example, in various embodiments the first stage is conducted in a vertical or horizontal pretreatment digester and the second stage is conducted in a suitable second vessel such as, for example, a flash tank.

In any of the various aspects, the lignocellulosic hydrolyzate is formed by contacting the impregnated lignocellulosic biomass with steam at a temperature of from about 150° C. to about 250° C., from about 150° C. to about 220° C., from about 175° C. to about 220° C., or from about from about 175° C. to about 200° C., such as about 175° C., about 180° C., about 185° C., about 190° C., about 195° C., or about 200° C., and a pressure of from about 55 psig (about 380 kPa gauge) to about 560 psig (about 3870 kPa gauge), from about 55 psig (about 380 kPa gauge) to about 320 psig (about 2220 kPa gauge), from about 55 psig (about 380 kPa gauge) to about 250 psig (1725 kPa gauge), from about 75 psig (518 kPa gauge) to about 225 psig (1550 kPa gauge), from about 90 psig (620 kPa gauge) to about 210 psig (1450 kPa gauge), from about 115 psig (about 790 kPa gauge) to about 320 psig (about 2220 kPa gauge), or from about 150 psig (1035 kPa gauge) to about 200 psig (1380 kPa gauge). Total elevated temperature and pressure contact time is typically from about 1 minute to about 60 minutes, from about 1 minute to about 30 minutes, from about 1 minute to about 10 minutes, or from about 2 minutes to about 6 minutes. After the contact time has elapsed, the pressure is reduced to less than about 25 psig (173 kPa gauge), such as about 20 psig (138 kPa gauge), about 17.5 psig (121 kPa gauge), about 15 psig (104 kPa gauge), about 12.5 psig (86 kPa gauge), about 10 psig (69 kPa gauge), about 7.5 psig (52 kPa gauge), about 5 psig (about 35 kPa gauge), about 4 psig (28 kPa gauge), or about 3 psig (21 kPa gauge), and ranges thereof, such as from about 3 psig to about 20 psig, from about 3 psig to about 15 psig, from about 3 psig to about 10 psig or from about 3 psig to about 5 psig, to form the steam pretreated insoluble biomass (i) in a single pressure reduction step or (ii) the pressure is reduced to from about 20 psig (140 kPa gauge) to about 100 psig (690 kPa gauge), from about 25 psig (175 kPa gauge) to about 75 psig (520 kPa gauge), from about 50 psig (345 kPa gauge) to about 75 psig (520 kPa gauge), from about 50 psig (345 kPa gauge) to about 200 psig (1380 kPa gauge), from about 50 psig to about 175 psig (1205 kPa gauge), from about 100 psig (690 kPa gauge) to about 200 psig, from about 100 psig to about 175 psig, from about 100 psig to about 150 psig (1035 kPa gauge), or from about 150 psig to about 175 psig in a first (intermediate) pressure reduction step and held for a period of time from about 0.5 minutes to about 30 minutes, from about 0.5 minutes to about 15 minutes, or from about 1 minute to about 5 minutes, followed by reduction to less than about 25 psig in a second step as described above.

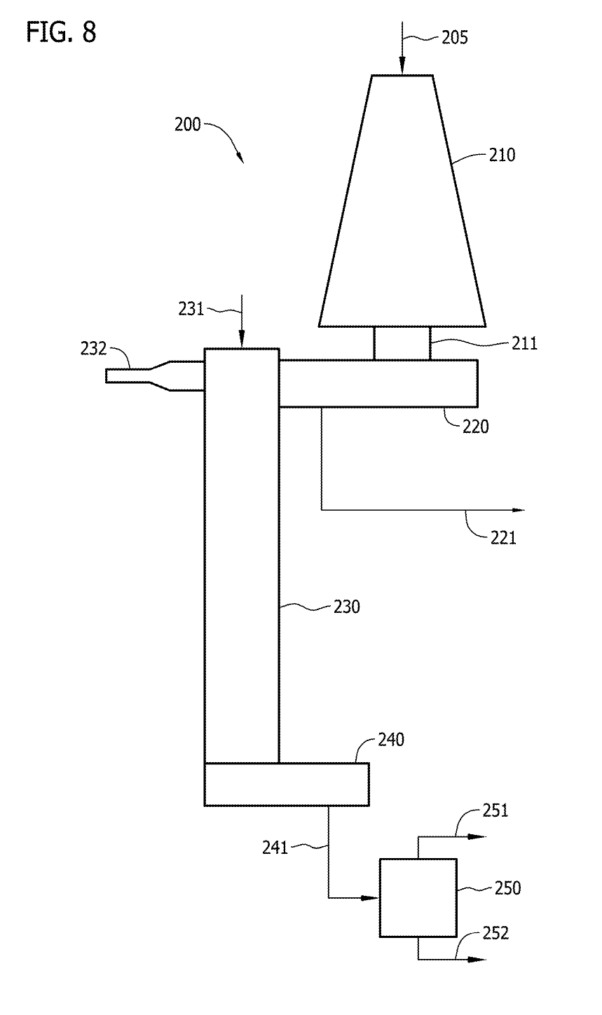

Referring now to FIG. 8, one non-limiting example of a steam explosion system 200 is depicted for steam explosion of dewatered lignocellulosic biomass material 205 wherein the system may include a feed vessel 210, a plug screw feeder 220 that transfers impregnated and dewatered lignocellulosic biomass 205 to a digester 230. The feed vessel 210 is suitably sized to provide sufficient storage capacity to allow acid impregnated and dewatered lignocellulosic biomass 205 to be introduced to the digester 230 at a relatively constant rate, and may have a cylindrical shape with a diverging wall (i.e., the diameter of the bottom is larger than the diameter of the top), but may alternatively have another suitable shape. In some aspects of the disclosure, the acid concentration or acidity (e.g., pH) of the liquid effluent 221 discharged from the plug screw feeder 220 is measured. In some other aspects of the disclosure, a metering device 211 feeds dewatered lignocellulosic biomass 205 from the feed vessel 210 to the plug screw feeder 220. In acid impregnation embodiments, the acid concentration may be adjusted and thereby controlled by increasing or decreasing the concentration of acid in the acid stream that is used as a source of acid during acid impregnation. In some aspects, the pH of the liquid effluent 221 is controlled to be between about 1 and about 2. Steam 231 is introduced to digester 230. In some aspects, the mass ratio of steam 231 to dewatered lignocellulosic biomass 205 (based on dry biomass) added to the vessel is at least about 1:6 or, as in other embodiments, at least about 1:4 or at least about 1:1.5 and is controlled by metering device 211. The temperature within the digester 230 (and of the biomass after sufficient residence time) may be controlled to temperature ranges as describe elsewhere herein by, for example, controlling the pressure of steam 231 introduced into the vessel and/or venting steam through vent 232.

In further reference to FIG. 8, steam pretreated lignocellulosic biomass may be evenly removed from the bottom of the digester 230 by use of a rotary sweeper (not shown) positioned above a screw conveyor 240 which conveys the treated lignocellulosic biomass material 241 through a blow valve assembly (not shown) and into a flash tank 250 (blow tank) in which steam 251 is flashed from the biomass and discharged. The flash tank 250 may receive material from one pretreatment digester or from two or more digesters without limitation. Pretreated lignocellulosic biomass hydrolyzate 252 is then discharged from flash tank 250.

The lignocellulosic hydrolyzate slurry is typically characterized by a total solids content of from about 20 wt. % to about 60 wt. %, from about 30 wt. % solids to about 50 wt. %, from about 35 wt. % to about 50 wt. %, or from about 40 wt. % to about 50 wt. %. The slurry pH suitably varies with the pH of the impregnated lignocellulosic biomass and may be in the range of from about 1 to about 5, or from about 1 to about 4, or from about 8 to about 12, or from about 9 to about 11. The insolublesolids fraction of the pretreated lignocellulosic feedstock generally comprises cellulose, unsolubilized lignin, unsolubilized hemicellulose, and unsolubilized ash. The composition of the insoluble solids fraction of the pretreated feedstock generally corresponds to the composition of the acid-impregnated feedstock, adjusted for break-down of the cellulose-hemicellulose-lignin complex. Typically, cellulose constitutes from about 35 wt. % to about 65 wt. %, from about 40 wt. % to about 60 wt. % or from about 45 wt. % to about 55 wt. % of the solids fraction. The insoluble solids fraction further typically comprises from about 20 wt. % to about 40 wt. % or from about 20 wt. % to about 30 wt. % lignin and from about 4 wt. % to about 10 wt. % hemicellulose. The liquid fraction of the pretreated lignocellulosic feedstock generally comprises solubilized hemicellulose, solubilized cellulose, oligomers, polysaccharides, monosaccharides and solubilized components provided by degradation of lignin. Fermentable sugars (e.g., glucose, xylose, arabinose, mannose, galactose, and various oligomers thereof) generally comprise from about 50 wt. % to about 95 wt. % or from about 60 wt. % to about 90 wt. % of the water-soluble components of the liquid fraction, and lignin and lignin degradation compounds generally comprise from about 0.5 wt. % to about 5 wt. % of the water-soluble components of the liquid fraction.

In any of the various aspects, in general, the lignocellulosic hydrolyzate pH and temperature are adjusted to form an adjusted lignocellulose hydrolyzate slurry having a high solids content. The adjusted lignocellulosic hydrolyzate slurry pH and temperature are adjusted to a range suitable for enzymatic activity, such as a pH of from about 4 to about 6.5, from about 4.5 to about 6, or from about 5 to about 5.5, and a temperature of from about 30° C. to about 70° C., from about 35° C. to about 70° C., from about 45° C. to about 65° C., from about 30° C. to about 60° C., from about 40° C. to about 60° C., or from about 50° C. to about 55° C. The slurry solids content is from about 20 wt. % to about 40 wt. % or from about 30 wt. % to about 35 wt. %, such as about 30 wt. %, about 31 wt. %, about 32 wt. %, about 33 wt. %, about 34 wt. %, about 35 wt. %, about 36 wt. %, about 37 wt. %, about 38 wt. %, about 39 wt. % or about 40 wt. %. In some aspects of the present disclosure, the lignocellulosic hydrolyzate is purified to remove enzymatic and/or fermentation inhibitors.

In some aspects, pretreated lignocellulosic biomass is contacted with steam at a treatment pressure and temperature of about 150 psig and about 185° C., of about 160 psig and about 188° C., about 170 psig and about 191° C., about 180 psig and about 193° C., about 190 psig and about 195° C. or about 200 psig and about 198° C. and ranges thereof, such as from about 150 psig (185° C.) to about 200 psig (198° C.), from about 160 psig (188° C.) to about 180 psig (193° C.) or from about from about 160 psig (188° C.) to about 170 psig (191° C.). The treated lignocellulosic biomass is depressurized to a flash tank pressure of from about 12 psig to about 17 psig, from about 13 psig to about 16 psig, or from about 14 psig to about 16 psig resulting in a lignocellulosic hydrolyzate temperature of about 119° C., about 119.5° C., about 120° C., about 120.5° C., about 121° C., about 121.5° C., about 122° C., about 122.5° C. or about 123° C., and ranges thereof, such as from about 119° C. to about 123° C. or from about 120° C. to about 122° C. In such aspects, the flashed condensate is from about 12 wt. %, about 12.5 wt. %, about 13 wt. %, about 13.5 wt. % or about 14 wt. %, and ranges thereof, such as from about 12 wt. % to about 14 wt. % or from about 13 wt. % to about 14 wt. % of the original mass of water in the steam pretreated acid impregnated lignocellulosic biomass, and the steam pretreated impregnated lignocellulosic biomass (e.g., acid impregnated biomass) after condensate flash comprises about 42.5 w. % solids, about 43 wt. % solids, about 43.1 wt. % solids, about 43.2 wt. % solids, about 43.3 wt. % solids, about 43.4 wt. % solids, about 43.5 wt. % solids, about 43.6 wt. % solids or about 43.7 wt. % solids, and ranges thereof, such as between about 42.5 and about 43.7 wt. % solids or between about 43 and about 43.7 wt. % solids. In one particular aspect, pretreated lignocellulosic biomass is contacted with steam at a treatment pressure and temperature of about 167 psig (1152 kPa gauge) and about 190° C. and is depressurized to about 15 psig (about 103.5 kPa gauge) resulting in (i) an impregnated lignocellulosic biomass having a temperature of about 121° C., (ii) flashed condensate of from about 13 wt. % to about 14 wt. % of the original mass of water in steam pretreated acid impregnated lignocellulosic biomass, and (iii) steam pretreated acid impregnated lignocellulosic biomass (after condensate flash) comprising between about 43 wt. % and about 43.7 wt. % total solids. In one optional aspect, the treated lignocellulosic biomass is depressurized to a flash tank pressure of atmospheric pressure and a temperature of about 100° C.

In some other aspects, pretreated lignocellulosic biomass is contacted with steam at a treatment pressure and temperature as disclosed above. The treated lignocellulosic biomass is depressurized to a flash tank pressure range of from about 4 psig to about 8 psig, from about 4 psig to about 7 psig, or from about 4 psig to about 6 psig, resulting in a lignocellulosic hydrolyzate temperature of about 106.9° C., about 107.6° C., about 108.5° C., about 109.1° C., about 109.9° C., about 110.6° C., about 111.2° C., about 111.9° C. or about 112.6° C., and ranges thereof, such as from about 106.9° C. to about 112.6° C., from about 106.9° C. to about 111.2° C. or from about 106.9° C. to about 109.9° C. In such aspects, the flashed condensate is about 14 wt. %, about 14.5 wt. %, about 15 wt. %, or about 15.5 wt. %, and ranges thereof, such as from about 14 wt. % to about 15.5 wt. %, of the original mass of water in the steam pretreated acid impregnated lignocellulosic biomass, and the steam pretreated impregnated lignocellulosic biomass (e.g., acid impregnated biomass) after condensate flash comprises about 43.7 w. % solids, about 43.8 wt. % solids, about 43.9 wt. % solids, or less than about 44 wt. % solids, and ranges thereof, such as between about 43.7 and about 44 wt. %. In one particular aspect, pretreated lignocellulosic biomass is contacted with steam at a treatment pressure and temperature of about 167 psig (1152 kPa gauge) and about 190° C. and is depressurized to about 5 psig (about 34.5 kPa gauge) resulting in (i) an impregnated lignocellulosic biomass having a temperature of about 108.5° C., (ii) flashed condensate of from about 14 wt. % to about 15.5 wt. % of the original mass of water in steam pretreated acid impregnated lignocellulosic biomass, and (iii) steam pretreated acid impregnated lignocellulosic biomass (after condensate flash) comprising between about 43.7 wt. % and about 44 wt. % total solids.

In some other aspects, pretreated lignocellulosic biomass is contacted with steam at a treatment pressure and temperature as disclosed above. The treated lignocellulosic biomass is depressurized to a flash tank pressure of about 2 psig, about 2.5 psig, about 3 psig or about 3.5 psig, and ranges thereof, such as from about 2 psig to about 3.5 psig, or from about 2.5 psig to about 3.5 psig, resulting in a lignocellulosic hydrolyzate temperature of about 103.6° C., about 104.4° C., about 105.3° C. or about 106.1° C., and ranges thereof, such as from about 103.6° C. to about 106.1° C., or from about 104.4° C. to about 106.1° C. In such aspects, the flashed condensate is about 16 wt. %, about 16.5 wt. %, about 17 wt. %, or about 17.5 wt. %, and ranges thereof, such as from about 16 wt. % to about 17.5 wt. % or from about 16 wt. % to about 17 wt. % of the original mass of water in the steam pretreated acid impregnated lignocellulosic biomass, and the steam pretreated impregnated lignocellulosic biomass (e.g., acid impregnated biomass) after condensate flash comprises about 44 wt. % solids, about 44.2 wt. % solids, about 44.6 wt. % solids, about 44.8 wt. % solids or about 45 wt. % solids, and ranges thereof, such as from about 44 wt. % to about 45 wt. % solids. In one particular aspect, pretreated lignocellulosic biomass is contacted with steam at a treatment pressure and temperature of about 167 psig (1152 kPa gauge) and about 190° C. and is depressurized to about 5 psig (about 34.5 kPa gauge) resulting in (i) an impregnated lignocellulosic biomass having a temperature of about 105° C., (ii) flashed condensate of from about 16 wt. % to about 17 wt. % of the original mass of water in steam pretreated acid impregnated lignocellulosic biomass, and (iii) steam pretreated acid impregnated lignocellulosic biomass (after condensate flash) comprising from about 44 wt. % and about 45 wt. % total solids

In a one adjustment option of the present disclosure, the steam pretreatment step contact pressure and temperature and flash tank pressure are such that the resulting lignocellulosic hydrolyzate is characterized by a temperature in excess at least 105° C., at least 106° C., at least 107° C. or at least 108° C., such as from about 105° C. to about 130° C., from about 105° C. to about 125° C., from about 105° C. to about 120° C., from about 106° C. to about 125° C., from about 107° C. to about 125° C. or from about 108° C. to about 125° C., and the lignocellulosic hydrolyzate is discharged from the flash tank to an extractor where it is contacted with a chilled and pH-adjusted aqueous wash stream to form an adjusted slurry having an adjusted pH, reduced temperature, and a reduced concentration of soluble compounds such as monosaccharides and inhibitors.

In reference to FIGS. 1, 2 and 4 to 6, pretreated lignocellulosic biomass hydrolyzate 15 is processed in extractor 30 by contact with a wash fluid comprising chilled and pH adjusted water 31 to form an adjusted extracted lignocellulosic biomass slurry 35 and a rich liquor stream 36 that is enriched in xylose as compared to the adjusted extracted lignocellulosic biomass slurry 35. In any of the various aspects, the aqueous wash stream has a temperature of less than about 15° C., less than about 10° C. or less than about 5° C. and a pH suitable for adjusting the pH of lignocellulosic hydrolyzate to a range suitable for enzymatic hydrolysis. For instance, for acid impregnated biomass, the aqueous wash stream suitably has a pH in the range of from about 8 to about 12, or from about 9 to about 11; for alkali impregnated biomass, the aqueous wash stream suitably has a pH in the range of from about 2 to about 5. Suitable weight ratios of wash stream to lignocellulosic hydrolyzate include about 8:1, about 7:1, about 6:1, about 5:1, about 4:1, about 3:1 or about 2:1, and ranges thereof, such as from about 2:1 to about 8:1, from about 5:1 to about 2:1 or from about 4:1 to about 2:1. Alternatively stated, the liquid to insoluble ratio for extraction is from about 1.5:1 to about 4:1, from about 2:1 to about 3:1, or from about 2:1 to about 2.5:1, such as about 1.5:1, about 2:1, about 2.5:1, about 3:1, about 3.5:1 or about 4:1. In any of the various extraction aspects, the total solids concentration in the adjusted slurry is less than 25 wt. %, less than about 22 wt. %, less than about 20 wt. %, less than about 15 wt. %, less than about 12 wt. %, less than about 10 wt. %, between about 5 wt. % and about 25 wt. %, from about 5 wt. % to about 22 wt. %, from about 5 wt. % to about 20 wt. %, from about 5 wt. % to about 15 wt. %, from about 10 wt. % to about 15 wt. %, or from about 10 wt. % to about 20 wt. % and the temperature is than about 70° C., less than about 60° C., less than about 50° C., from about 30° C. to about 70° C., from about 40° C. to about 60° C., from about 50° C. to about 55° C., or from about 50° C. to about 60° C., and the pH is from about 4 to about 7, from about 4 to about 6 or from about 4.5 to about 6.

Suitable extractors include inclined screw thickeners and diffusers. Inclined screw thickeners are known in the art and are available from, for instance, Kadant Black Clawson. In general, lignocellulosic hydrolyzate is fed to the thickener and contacted in a countercurrent fashion with the chilled pH-adjusted aqueous (wash) stream as it is conveyed upward. The pH and temperature of the biomass are adjusted as it contacts the wash stream and moves upward in the thickener where it is discharged as an adjusted slurry. The wash stream exits the diffuser at the bottom as a rich liquor stream enriched in extracted soluble compounds such as monosaccharides and inhibitors. Diffusers and known in the art and are available, for instance, from Maguin Company, and include a multi-cell counter-current diffuser, a diffusion tower, or a continuous beet diffuser. In diffuser operation, lignocellulosic biomass hydrolyzate and the chilled pH-adjusted aqueous (wash) stream are contacted in a counter-current scheme wherein the biomass is fed into the diffuser at lower elevation than the wash water. The biomass moves against the flow of the aqueous stream and is carrier upward where it is discharged as an adjusted slurry and the wash stream exits the diffuser at the bottom as a rich liquor stream enriched in extracted soluble compounds such as monosaccharides and inhibitors. In any of the aspects, the slurry pH may be controlled by adjusting the pH of the wash stream and/or by adding additional acid or base to the extractor. In any the various aspects, the adjusted slurry is essentially homogeneous in temperature and pH and comprises a reduced concentration of soluble compounds as compared to the lignocellulosic hydrolyzate. As used herein, essentially homogeneous temperature refers to a temperature of any portion of a referenced lignocellulosic biomass stream that does not vary by more than 10% from the temperature of any other portion of the lignocellulosic biomass stream, and essentially homogeneous pH refers to a pH of any portion of a referenced lignocellulosic biomass stream that does not vary by more than 10% from the pH of any other portion of the lignocellulosic biomass stream.

The rich liquor stream may be collected in a rich liquor tank. The rich liquor typically comprises from about 50 wt. % to about 90 wt. % or from about 60 wt. % to about 80 wt. % of the total soluble compounds present in the lignocellulosic hydrolyzate, such as about 50 wt. %, about 60 wt. %, about 70 wt. %, about 80 wt. % or about 90 wt. %. Soluble compounds include C5 monosaccharides and inhibitor compounds. In some aspects of the disclosure, the rich liquor tank is jacketed and agitated, and supplied with a cooling fluid for reducing the rich liquor temperature. At least a portion of the rich liquor may optionally be purged from the process.

The rich liquor may be optionally processed. For instance, the rich liquor pH may be adjusted by the addition of an acid or base. In some aspects, the pH of the rich liquor is adjusted to from about 4 to about 9, from about 4 to about 8 or from about 4 to about 6.5 by the addition of a suitable base, such as ammonia or sodium hydroxide. The rich liquor may optionally be purified to remove an inhibitor fraction therefrom. In one purification method, an acidic rich liquor may be admixed with a base such as sodium hydroxide, ammonium hydroxide or calcium oxide to form insoluble phenolic compounds, such as phenate salts, that may be removed from the rich liquor by means known in the art, such as filtration. In another purification method, phenolics may also be removed by contacting the rich liquor with activated charcoal. This method is of particular utility because hydrolyzate sugar levels are generally unaffected. In yet another method, furfurals, aliphatic carboxylic acids and phenolics may be removed by contact with a cationic or anionic ion exchange resin. In another method, the rich liquor may be extracted with ethyl acetate to remove furfurals, aliphatic carboxylic acids and phenolics. In still another method, the rich liquor may be stripped by stream injection and/or evaporated under vacuum to remove volatile organic inhibitors such as acetic acid, furfural and vanillin. Yet further, impurities may be removed by membrane filtration or extraction, such as by the use of adsorptive microporous membranes substituted with functional to which inhibitors bind. Combinations of one or more of the above purification methods are within the scope of the present disclosure.

In some aspects of the disclosure, inhibitors are not removed from the rich liquor. In such aspects, the inhibitor concentration is greater at 30 wt. % solids as compared to the inhibitor concentration at 20 wt. % solids in prior art processes. As described elsewhere herein, the fermentation organism may be adapted to such higher impurity levels. Advantageously, the inhibitors inhibit the growth and proliferation of unwanted bacteria while the growth and proliferation of adapted fermentation organisms are not significantly inhibited. Thus, fermentation yield may be essentially maintained at higher impurity levels while bacteria contamination may be inhibited thereby reducing the requirement for process sterilization, such as use of clean-in-place systems.

The rich liquor is passed through a cooler, such as a heat exchanger (plate and frame or tube design), to reduce the temperature. In some aspects of the disclosure, as described in more detail herein, at least a portion of the cooled rich liquor may optionally be combined with enzymatically hydrolyzed lignocellulosic biomass. In such aspects, the temperature is suitably less than about 60° C. In some other aspects of the disclosure, as described in more detail herein, at least a portion of the cooled rich liquor may be recycled to the flash tank and combined therein with the lignocellulosic hydrolyzate for partial temperature and pH adjustment thereof. In such aspects, the temperature is suitably no more than 20° C., less than about 15° C., less than about 10° C., about 5° C., from about 5° C. to about 20° C., from about 5° C. to about 15° C. or from about 5° C. to about 10° C.

The adjusted slurry is concentrated in a dewaterer to form dewatered lignocellulosic biomass and lean liquor (squeezate). Suitable dewaterers are known in the art and include mechanical solid/liquid separation devices such as a dewatering screw press. In any of the various aspects of the present disclosure, the dewatered lignocellulosic biomass has a total solids concentration of at least 25 wt. % or at least 30 wt. %, between about 25 wt. % to about 35 wt. %, from about 30 wt. % to about 35 wt. %, such as about 25 wt. %, about 26 wt. %, about 27 wt. %, about 28 wt. %, about 29 wt. %, about 30 wt. %, about 31 wt. %, about 32 wt. %, about 33 wt. %, about 34 wt. % or about 35 wt. %. The temperature of the dewatered lignocellulosic biomass is from about 30° C. to about 70° C., from about 40° C. to about 60° C., from 45° C. to about 55° C., or from about 50° C. to about 55° C., such as about 40° C., about 45° C., about 50° C., about 55° C. or about 60° C. As described elsewhere herein in more detail, the dewatered lignocellulosic biomass is contacted with a source of enzymes comprising cellulase to form a lignocellulosic biomass hydrolyzate. The lean liquor may be collected in a lean liquor hold tank that, in some aspects, is jacketed and agitated, and supplied with a cooling fluid for reducing the lean liquor temperature. The lean liquor comprises soluble compounds including monosaccharides and inhibitor compounds wherein the concentration thereof is less than the concentration of such soluble compounds in the rich liquor. For instance, the concentration of soluble compounds in the lean liquor is typically less than 30%, less than 20%, less than 10% of the concentration present in the rich liquor, and ranges thereof, such as from about 5 wt. % to about 30 wt. %, from about 5 wt. % to about 20 wt. %, from about 10 wt. % to about 30 wt. %, or from about 10 wt. % to about 20 wt. %. The lean liquor may be optionally processed. For instance, as depicted in FIGS. 2, 4 and 5, the lean liquor pH may be adjusted by the addition of a chilled and pH adjusted water stream. The lean liquor may optionally be purified or processed by solid liquid separation to remove suspended solids and/or an inhibitor fraction therefrom according to the methods described herein in connection with the rich liquor stream.

The lean liquor is passed through a cooler, such as a plate and frame heat exchanger (plate and frame or tube design), to reduce the temperature. In some aspects of the disclosure, as described in more detail herein, at least a portion of the cooled lean liquor may optionally be recycled to the extractor as wash water. In some other aspects of the disclosure, as described in more detail herein, at least a portion of the cooled lean liquor may be recycled to the flash tank and combined therein with the lignocellulosic hydrolyzate for partial temperature and pH adjustment thereof. In any such aspects, the lean liquor temperature is suitably less than about 15° C., less than about 10° C. or less than about 5° C.

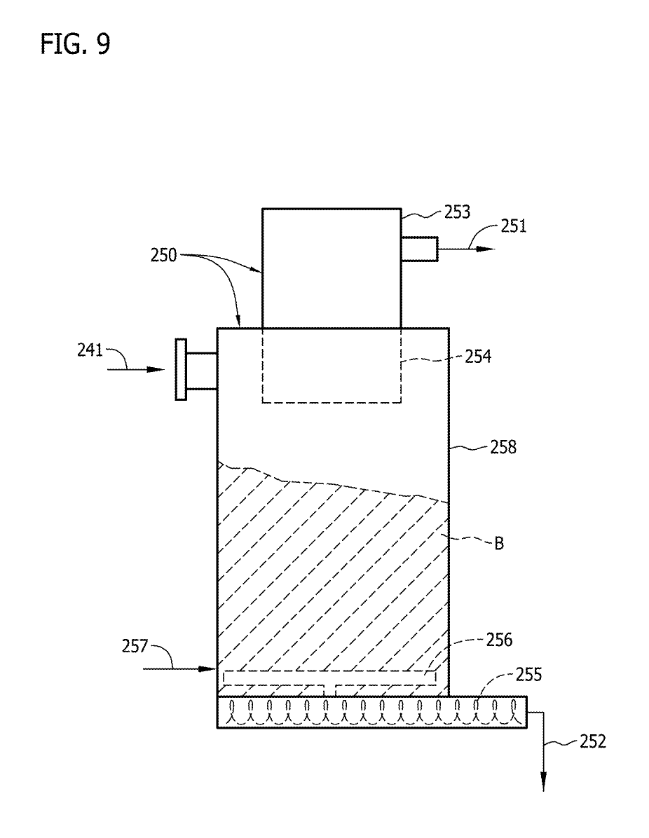

In some other adjustment aspects of the present disclosure, as depicted without limitation in FIGS. 4 to 6, chilled and pH adjusted water 21, chilled rich liquor 85, or a combination thereof, may be combined with the lignocellulosic hydrolyzate in the flash tank. For instance, as depicted in FIG. 9, cooling fluid 257 may comprise chilled and pH adjusted water 21, chilled rich liquor 85, or a combination thereof, and may be added to the flash vessel 250. Addition of such a stream to the flash tank provides for preliminary pH and temperature adjustment and assist in conveying the lignocellulose hydrolyzate from the flash tank to the extractor.

Referring to FIG. 9, in some non-limiting aspects of the disclosure, the flash vessel 250 may include a main section 258 wherein a bed B of received treated lignocellulosic biomass material 241 forms. The flash vessel 250 also includes a stand pipe 253 through which flashed steam 251 is removed. The stand pipe section 253 has a sufficiently large diameter such that the velocity of the steam 251 removed from the main section 258 is below the terminal velocity (i.e., entrainment velocity) of biomass material 241. Below the stand pipe 253 is a vortex finder 254 with a diameter the same as the diameter of the stand pipe and which extends below the entry point of biomass material 241 in the main section 258. In some embodiments (not shown), the diameter of the lower section of the vortex finder 254 may be larger than the diameter of its top section to enhance settling of solid particles. The ratio of the diameter of the stand pipe to the diameter of the flash vessel may be from about 1:5 to about 1:1.3 or, as in other embodiments, from about 1:3 to about 1:1.5. The ratio of the height of the vortex finder to the diameter of the flash vessel may be from about 1:2.5 to about 1:1.3 or from about 1:2 to about 1:1.4

In some aspects, the average residence time of steam 251 in the flash vessel 250 exceeds the time for at least about 90% of the biomass particles to reach near equilibrium (e.g., within about 5° C., within about 3° C. or within about 1° C. of equilibrium) with the liquor in the vessel. The time for at least about 90% of the biomass particles to reach near equilibrium with the liquor may vary with the size of the particles and the pressure of the flash vessel 250. In some embodiments, the average residence time of flash steam is at least about 1 second, at least about 2 seconds or at least about 4 seconds (e.g., from about 1 second to about 10 seconds or from about 2 seconds to about 6 seconds). The residence time of flash steam may be determined from the flash steam flow in the exhaust pipe connected to the stand pipe 253 and the volume of vapor space in the flash vessel 250 and stand pipe 253 (calculated based on a measured level).

In further reference to FIG. 9, an extraction screw 255 extends into the main section 258 of the flash vessel 250 to remove pretreated biomass material 252. In some aspects, the extraction screw 255 may suitably be a pair (or more) of twin screw conveyors in which the two screws turn in opposite directions. In some other aspects, the extraction screw 255 may have mixing paddles or cut flights to mix cooling fluid with the pretreated biomass as described below. An agitator 256 having a plurality of blades is located above the screw conveyor to mix the lignocellulosic hydrolyzate and distribute it onto the extraction screw 255 for removal from the main section 258 of the vessel. In any of the various aspects, a minimum biomass bed height (e.g., about 1 meters) is maintained in the vessel 250 to prevent flash steam from blowing through the bottom outlet of the flash tank through the screw conveyor and the blow valve assembly. The pretreated biomass in the flash vessel 250 may further be maintained at a level that ensures sufficient residence time of flash steam and below the inlet of the vortex finder 254 (e.g., at least about 1 meter below the inlet of the vortex finder).

In further reference to FIG. 9, in some optional aspects of the present disclosure, a cooling fluid 257 may be added to the vessel 250 to cool the biomass and adjust biomass total solids content (and, as in some embodiments, to adjust biomass pH as described below) to prepare the biomass for subsequent enzyme hydrolysis and fermentation. The cooling fluid 257 may be added through one or more injection nozzles (not shown) that extend through the vessel 250. In some embodiments of the present disclosure, the cooling fluid 257 is added to the vessel 250 at a point opposite the bed of biomass (i.e., not above or below the bed). The cooling fluid 257 may suitably be added at or above the agitator 256 to promote mixing of cooling fluid 257 throughout the biomass 241, such as from about 5 cm to about 50 cm or from about 10 cm to about 30 cm above the agitator blades. The cooling fluid 257 may also be added opposite the outlet of the screw conveyor 255 to promote mixing between the cooling fluid and the biomass before removal from the vessel 250.

Various equipment arrangement schemes are within the scope of the various extractor aspects of the present disclosure. For instance, and without limitation, in one scheme generally depicted in FIGS. 1, 2 and 4, one extractor (e.g., diffuser) may be used in combination one dewaterer (e.g., screw press). In another scheme, generally depicted in FIG. 5, two extractors and two dewaterers are arranged in parallel. In yet another scheme, generally depicted in FIG. 6, two extractors are arranged in parallel and feed a single dewaterers. Other arrangements are within the scope of the present disclosure. For instance, two extractors could be operated sequentially in combination with one or more dewaterers.

In one aspect of the present disclosure, depicted in FIG. 3, the pretreated lignocellulosic biomass is depressurized to a flash tank 20 pressure of about 3 psig (about 21 kPa gauge) or less resulting in a lignocellulosic hydrolyzate 12 having a temperature of no more than about 105° C., no more than about 106° C., no more than about 107° C. or no more than about 108° C. Chilled and pH adjusted water 21 is added to the flash tank as described elsewhere herein to form cooled and pH adjusted pretreated lignocellulosic biomass 22 having a solids content of at least 30 wt. %, such as 30 wt. %, about 31 wt. %, about 32 wt. %, about 33 wt. %, about 34 wt. %, about 35 wt. %, about 36 wt. %, about 37 wt. %, about 38 wt. %, about 39 wt. %, or about 40 wt. %. The pretreated lignocellulosic biomass 22 is contacted with additional chilled and pH adjusted water 121 in dynamic mixer 120 to form adjusted pretreated lignocellulosic biomass 125 having a pH of from about 4 to about 6, a temperature of from about 30° C. to about 70° C., from about 40° C. to about 60° C., form about 50° C. to about 60° C., or from about 50° C. to about 55° C., and a total insoluble solids content of at least 25 wt. %, such as about 25 wt. %, about 26 wt. %, about 27 wt. %, about 28 wt. %, about 29 wt. %, or about 30 wt. %. It has been discovered that, for lignocellulosic hydrolyzates having a temperature of no more than about 105° C., no more than about 106° C., no more than about 107° C. or no more than about 108° C., sufficient cooling and pH adjustment can be achieved utilizing such a two-stage adjustment scheme without the further requirement of washing in an extractor.

The dewatered and adjusted lignocellulosic biomass is transferred from the dewaterer to enzymatic hydrolysis, such as by a conveyor, to enzymatic hydrolysis where it is combined with a source of enzymes comprising cellulase to hydrolyze cellulose and produce fermentable sugars. As is known in the art, cellulases are a class of enzymes produced chiefly by fungi, bacteria, and protozoans that catalyze the hydrolysis of cellulose into glucose, cellobiose, cellotriose, cellotetrose, and longer chain cellodextrins. Cellulase includes both exohydrolysase and endohydrolysases that are capable of recognizing cellulose, or cellodextrins, as substrates. Cellulase enzymes may include endoglucanases, cellobiohydrolysases, beta-glucosidases, alone or in combination. The source of enzymes may further comprises at least one of a hemicellulase (to hydrolyze hemicellulose to soluble pentose sugars (e.g., xylose)), an α-amylase (to liquefy free starch that was formerly entrapped within the cellulose, hemicellulose and/or lignocellulosic matrices), a β-amylase, a glucoamylase (to convert liquefied starch to C6 sugars), an arabinoxylanase, a pullulanase, and/or a protease (to hydrolyze peptide bonds and release starch granules encased in the protein matrix) for the purpose of generating additional hexose and pentose sugars. In some aspects of the disclosure, the lignocellulosic hydrolyzate slurry may be contacted with a source of enzymes comprising cellulase in a first hydrolysis step and then contacted with a source of enzymes comprising a glucoamylase in a second hydrolysis step

Cellulase loading in the slurry may suitably vary with the cellulose content, but typical loading may be expressed as from about 5 mg to about 50 mg enzyme protein per gram of cellulose, from about 10 mg to about 50 mg enzyme protein per gram of cellulose, from about 20 mg to about 50 mg enzyme protein per gram of cellulose, from about 10 mg to about 50 mg enzyme protein per gram of cellulose, from about 10 mg to about 40 mg enzyme protein per gram of cellulose, from about 10 mg to about 30 mg enzyme protein per gram of cellulose, from about 20 mg to about 50 mg enzyme protein per gram of cellulose or from about 20 mg to about 40 mg enzyme protein per gram of cellulose.

Cellulase may be combined with the treated biomass slurry by any means known in the art to achieve a substantially homogeneous admixture, including agitated mixing tanks, in line mixers, pug mill mixers, paddle mixers, ribbon mixers, or in liquefaction reactors such as reactors having at least one mixing section and at least one plug flow section. The enzymatic hydrolysis reactor is typically an agitated vessel designed to hold the treated biomass slurry-cellulase mixture at a temperature suitable for cellulose hydrolysis by cellulase, wherein the volume is sufficient to provide a hold time required for a significant yield of cellulose-derived hexose monosaccharide (“C6”) sugars, e.g., glucose. In some aspects of the present disclosure, the enzymatic hydrolysis vessel may be insulated and/or heated with a heating jacket to maintain hydrolysis temperature. Total enzymatic hydrolysis cycle times of 48 hours, 54 hours, 60 hours, 66 hours, 72 hours, 78 hours, 96 hours and 144 hours, and ranges thereof, are within the scope of the present disclosure. Glucose yields, based on total cellulose content of the biomass slurry is typically from about 30% to about 90%, from about 40% to about 80% from about 30% to about 70% or from about 60% to about 75% of the theoretical value.

For highly viscous treated biomass slurries, such as having a viscosity in excess of about 20,000 cP, about 30,000 cP, about 50,000 cP, about 60,000 cP or even about 100,000 cP, mixing with enzymes can be done in two stages. In a first stage, cellulase can be admixed with the biomass in a mixer particularly suited for the processing of highly viscous materials, for instance, a pug mill mixer, a paddle mixer (single or double shaft), or a ribbon mixer (single or double shaft). High viscosity mixers are particularly suited to the method of the present disclosure because thorough mixing of cellulase with the viscous treated biomass slurry enables a rapid viscosity reduction in the subsequent liquefaction step where the viscosity is preferably reduced to less than about 20,000 cP, less than about 15,000 cP, less than about 10,000 cP or even less than about 5,000 cP. The high viscosity mixer may optionally have a jacket to receive cooling or heating medium in order to maintain the temperature of the treated biomass during cellulase addition. Optionally, cooling and heating medium may be incorporated into the internal mixer components (such as rotating shafts, paddles) to further enhance heat exchange. In some aspects, cellulase addition can be done through one or more addition points, for example, multiple spray nozzles, position near the treated biomass inlet. In a second stage, the treated biomass-cellulase admixture may be processed in a mix tank or fiber liquefaction bioreactor. In some aspects, the treated biomass-cellulase admixture may be processed in a fiber liquefaction bioreactor to further reduce the viscosity prior to transfer to a cellulose hydrolysis reactor. The fiber liquefaction bioreactor may be of either a continuous mixing design or a design having at least one continuous mixing section and at least one plug flow section. Optionally, two or more fiber liquefaction bioreactors may be operated in series. In some particular aspects, the fiber liquefaction bioreactor comprises alternating mixing zones and near plug flow zones and the treated biomass-cellulase admixture either flows downward through the tower by gravity or is moved upward by pumping. The treated biomass-cellulase admixture is typically processed in a fiber liquefaction bioreactor until the admixture viscosity is less than about 10,000 cP, less than about 9,000 cP, less than about 8,000 cP, less than about 7,000 cP or less than about 5,000 cP where after it is transferred to a cellulose hydrolysis reactor.

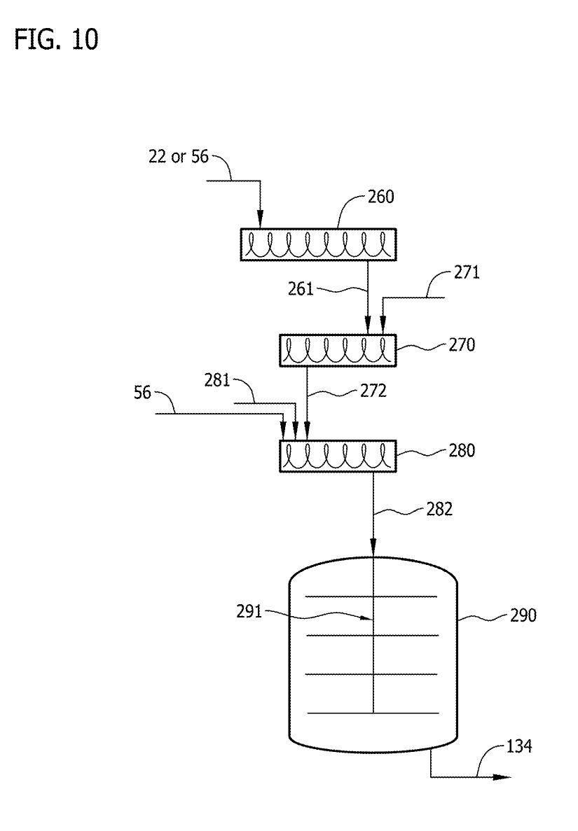

In aspects of the disclosure wherein the lignocellulosic hydrolyzate is not washed, as depicted in FIG. 10, the pretreated lignocellulosic biomass 22 is introduced into a mixing screw conveyor 260 which provides further mixing of biomass to uniformly cool the biomass. The mixing screw conveyor 260 may be water jacketed to provide further cooling. In such aspects of the disclosure, the temperature of the discharge slurry 261 is less than about 80° C., less than about 70° C., less than about 60° C., less than about 50° C., from about 30° C. to about 80° C., from about 50° C. to about 80° C., from about 30° C. to about 70° C., or from about 50° C. to about 70° C. The discharge slurry 261 from the mixing screw conveyor 260 is introduced into a neutralization mixer 270 wherein a chilled alkaline or acidic process stream 271 is added to the neutralization mixer 270 to provide and adjustment of pH, total solids content and temperature of the pretreated lignocellulosic biomass 22 before enzymatic hydrolysis and provide a conditioned lignocellulosic biomass 272. The pH adjustment in the neutralization mixer 270 may be a “fine” adjustment (e.g., 0.1 to 0.4 pH adjustment) with the crude adjustment occurring upstream in the flash vessel 250. The amount of pH adjustment may be controlled by changing the concentration of alkali or acid in process stream 271. After final pH adjustment, the pH of the neutralized biomass 270 may be, for example, from about 4.0 to about 5.5 or from about 4.8 to about 5.2 depending on the particular enzyme used for liquefaction.

In some optional aspects of the present disclosure, the dewatered lignocellulosic biomass 56 may be cooled and/or pH adjusted in a mixing screw conveyor 260 and/or a neutralization mixer 270 as described above in connection with the pretreated lignocellulosic biomass 22.

In some embodiments, the alkaline or acidic process stream 271 is cooled such as to a temperature less than about 25° C., less than about 20° C. or less than about 15° C. (e.g., from about 5° C. to about 25° C. or from about 10° C. to about 20° C.). The neutralization mixer 270 may be water jacketed to provide cooling. The neutralization mixer 270 may be any suitable mixing apparatus and may be a dynamic mixer such as a paddle mixer, pug mill mixer or helical mixer. The alkaline or acidic process stream 271 also provides final adjustment of the total solids content of the conditioned biomass. The amount of process stream 271 mixed with the biomass in the mixer may be adjusted based on total solids measurements made downstream of the neutralization mixer 270 (e.g., in the discharge) and upstream of the liquefaction bioreactor 290. Total solids content may be determined by sampling the material and measuring the mass of the sample before and after evaporating the moisture by heating (e.g., by infrared (IR) moisture balances). The total solids content may also be measured by near infrared (NIR) moisture analyzers or portable (e.g., handheld) moisture meters.

Dewatered lignocellulosic biomass 56 or conditioned lignocellulosic biomass 272 is introduced into an enzyme mixer 280. The enzyme mixer 280 may be a dynamic mixer (high viscosity mixer) such as a paddle mixer, pug mill mixer or other suitable mixing apparatus. Enzyme 281 is added to the mixer (e.g., enzyme dispersed through a liquid medium such as water) to begin enzymatic hydrolysis of the conditioned feedstock. The high viscosity mixer may optionally have a jacket to receive cooling or heating medium in order to maintain the temperature of the treated biomass during cellulase addition. Optionally, cooling and heating medium may be incorporated into the internal mixer components (such as rotating shafts, paddles) to further enhance heat exchange. In some aspects, cellulase addition can be done through one or more addition points, for example, multiple spray nozzles, position near the treated biomass inlet. In some embodiments, an enzyme 281 is added to cause downstream liquefaction of the biomass in which the conditioned biomass transitions from a high viscosity slurry to a pumpable low viscosity slurry. Suitable enzymes include, for example, cellulase, hemicellulase, pectinase, and lignin degrading enzyme.

After addition of enzyme, the enzyme-containing pretreated biomass slurry 282 is introduced into a liquefaction bioreactor 290 to partially hydrolyze the biomass thereby reducing the viscosity of the biomass (e.g., from a starting viscosity of about 20,000 cP or more to a reduced viscosity of about 5,000 cP or less) so that it can be pumped and processed in downstream saccharification operations or be subjected to solid/liquid separation. The liquefaction bioreactor 290 may be a plug-flow reactor with a suitable height-to-diameter ratio (e.g., of at least 2:1, at least about 3:1 or even about 4:1 or more). The average residence time through the liquefaction bioreactor 290 may be at least about 10 minutes, at least about 15 minutes, at least about 30 minutes or at least about 45 minutes (e.g., from about 10 to about 90 minutes or from about 15 to about 60 minutes).

The liquefaction bioreactor 290 may include, without being restricted to any particular design, an agitator 291 having multiple impellers that creates one or more mixing zones (e.g., 2, 3, 4 or 5 or more mixing zones) in the reactor. The top-most impeller may be positioned near the surface of the slurry to disperse the biomass across the liquefaction bioreactor 290. The hydraulic residence time in each mixing zone may be, for example, about 1 to about 10 minutes (e.g., about 1 to about 3 minutes). The height of each mixing zone may be from about 0.6 to about 1.2 meters. The mixing zones may be separated by an average hydraulic residence time of from about 2 to about 10 minutes. The impellers may be evenly spaced or unevenly spaced with the spacing being less near the upper portions of the reactor where slurry viscosity is relatively higher. Each impeller may be sized and shaped to provide a suitable mixing zone height. The impeller design and rotational speed of the agitator may be selected to provide radial mixing of the slurry with minimal vertical pumping action. The particular bioreactor dimensions, residence times and impeller designs described above are exemplary and any suitable dimensions, residence times and impeller designs may be used without limitation unless stated otherwise herein.

In some embodiments of the present disclosure, pH of the partially hydrolyzed pretreated biomass is measured in the liquefaction bioreactor 290. The measured pH may be used to provide fine pH control by adjusting one or both of (1) the amount of alkaline or acidic solution 271 introduced into the neutralization mixer 270 and (2) the concentration of alkali or acid in the alkaline solution 271. Alternatively or in addition, the measure pH may be used to provide coarse pH control by adjusting one or both of (1) the amount of alkaline or acidic solution (e.g., cooling fluid) introduced into the flash vessel 250 and/or the extractors 30 and 40 and (2) the concentration of alkali or acid in the solution added to the flash vessel based on the measured pH of the partially hydrolyzed pretreated biomass in the liquefaction bioreactor. The pH may be measured at any point (middle, top or bottom of the slurry) in the liquefaction bioreactor 290 including at the discharge of the liquefied slurry 134. In-line pH or conductivity meters may be used to continually monitor the pH of the slurry in the liquefaction bioreactor 290 or the pH of samples of slurry taken from the bioreactor may be measured by trained technicians or laboratory personnel. In some embodiments, the measured pH is used, at least partially, to adjust the concentration of alkali or acid in the cooling fluid 257.

As an alternative to, or in addition to, measurement of pH in the liquefaction bioreactor 290, the pH of the conditioned biomass 272, enzyme containing biomass 282, the dewatered lignocellulosic biomass 56, the rich liquor 36, 46 or 47, the lean liquor 55, 65 or 67, the contents of extractor 30 or 40, and combinations thereof, may be measured for pH feedback control in the neutralization mixer 270.

In some aspects of the present disclosure, pretreated biomass downstream of the flash tank 20 and upstream of the liquefaction bioreactor 290 is analyzed to provide feedback information for the extractors 30 and 40. For example, one or more of the following may be determined for feedback control of the pretreatment operations: pH, total solids, liquid fraction composition (e.g., sugars and inhibitors) and solid fraction composition (e.g., glucan, xylan and lignin). The water-insoluble traction of pretreated biomass may be monitored (intermittently or continuously, in-line or off-line) using near infrared (NIR) or FourierTransform NIR (FT-NIR) spectroscopy with multivariate analysis or by other suitable methods.

In some optional aspects of the present disclosure, a least a portion of the chilled pH adjusted rich liquor stream 85 may be combined with the liquefied slurry 134 in a mixer 140 to form monosaccharide rich stream 135 that is enriched in C5 monosaccharides as compared to the liquefied slurry stream 134. Any mixer suitable for operation at a viscosity of less than about 10,000 cP or less than about 5,000 cP is suitable for the practice of the present disclosure and includes, without limitation, in-line static mixers. In the case of rich liquor that has not been purified to remove inhibitors, the rich liquor is preferably combined with the liquefied slurry 134 instead of with the dewatered lignocellulosic biomass 56 in the lignocellulosic enzymatic hydrolysis step 130 so that the cellulase (and optionally other enzymes) are not subjected to elevated levels of inhibitors in the initial phase of hydrolysis such that a higher cellulose hydrolysis rate is achieved and/or a lower cellulase loading is required.

Any of the various enzyme treated biomass solid streams, slurry stream and aqueous streams may be utilized by suitable microorganisms as a substrate for the production of fermentation products. A wide variety of fermentation microorganisms are known in the art, and others may be discovered, produced through mutation, or engineered through recombinant means. Fermentation microorganisms within the scope of the present disclosure include yeast, bacteria, filamentous fungi, microalgae, and combinations thereof. Fermentation organisms may be wild type microorganisms or recombinant microorganisms, and include Escherichia, Zymomonas, Saccharomyces, Candida, Pichia, Streptomyces, Bacillus, Lactobacillus, Deinococcus and Clostridium. In some aspects of the present disclosure, the fermentation organism is recombinant Escherichia coli, Zymomonas mobilis, Bacillus stearothermophilus, Saccharomyces cerevisiae, Clostridia thermocellum, Thermoanaerobacterium saccharolyticum, Deinococcus or Pichia stipitis. Examples of fermentation products within the scope of the present disclosure include, for instance, acids, alcohols, alkanes, alkenes, aromatics, aldehydes, ketones, triglycerides, fatty acids, biopolymers, proteins, peptides, amino acids, vitamins, antibiotics, pharmaceuticals, and combinations thereof. Non-limiting examples of alcohols include methanol, ethanol, propanol, isopropanol, butanol, ethylene glycol, propanediol, butanediol, glycerol, erythritol, xylitol, sorbitol, and combinations thereof. Non-limiting examples of acids include acetic acid, lactic acid, propionic acid, 3-hydroxypropionic, butyric acid, gluconic acid, itaconic acid, citric acid, succinic acid, levulinic acid, and combinations thereof. Non-limiting examples of amino acids include glutamic acid, aspartic acid, methionine, lysine, glycine, arginine, threonine, phenylalanine, tyrosine, and combinations thereof. Other examples of fermentation products include methane, ethylene, acetone and industrial enzymes.

Hexose sugar fermenting organisms include yeasts. Any of a variety of yeasts can be employed as the yeast in the present method. Typical yeasts include any of a variety of commercially available yeasts, such as commercial strains of Saccharomyces cerevisiae. Suitable commercially available strains include ETHANOL RED (available from Red Star/Lesaffre, USA); BioFenn HP and XR (available from North American Bioproducts); FALI (available from Fleischmann's Yeast); SUPERSTART (available from Lallemand); GERT STRAND (available from Gert StrandAB, Sweden); FERMIOL (available from DSM Specialties); and Thennosac (available from Alltech). In some aspects, the hexose fermenting organism is a recombinant yeast having at least one transgene expressing an enzyme useful for converting mono- and/or oligo-saccharides to ethanol.

Suitable pentose sugar (e.g., xylose) fermenting organisms include yeasts. Such yeasts include Pachysolen tannophilus, Pichia stipitis, Candida diddensii, Candida utilis, Candida tropicalis, Candida subtropicalis, Saccharomyces diastaticus, Saccharomycopsis fibuligera and Torula candida. In some aspects, the pentose fermenting organism is a recombinant yeast having at least one transgene expressing an enzyme useful for converting mono- and/or oligo-saccharides to ethanol. For instance, the genome of P. stipitis may be incorporated into S. cerevisiae by a gene shuffling method to produce a hybrid yeast capable of producing bioethanol from xylose while retaining the ability to survive in high concentrations of ethanol.

In some aspects of the present disclosure, organisms capable of fermenting both hexose and pentose sugars are utilized to convert monosaccharides to ethanol. Typically, such organisms are strains of S. cerevisiae having transgenes encoding for one or more enzymes capable of converting pentose sugars to ethanol.

Selection of suitable fermentation conditions may suitably be done by those skilled in the art based on (i) the identity of the microorganism or combination of microorganisms, (ii) the characteristics of the fermentation substrate medium and (iii) the associated fermentation product. Fermentation may be aerobic or anaerobic. Single and multi-step fermentations are within the scope of the present disclosure. The fermentation substrate medium may be supplemented with additional nutrients required for microbial growth. Supplements may include, for example, yeast extract, vitamins, growth promoters, specific amino acids, phosphate sources, nitrogen sources, chelating agents, salts, and trace elements. Components required for production of a specific product made by a specific microorganism may also be included, such as an antibiotic to maintain a plasmid or a cofactor required in an enzyme catalyzed reaction. Also additional sugars may be included to increase the total sugar concentration. Suitable fermentation conditions are achieved by adjusting these types of factors for the growth and target fermentation product production by a microorganism. The fermentation temperature can be any temperature suitable for growth and production of the nutrients of the present disclosure, such as from about 20° C. to about 45° C., from about 25° C. to about 40° C., or from about 28° C. to about 32° C. The fermentation pH can be adjusted or controlled by the addition of acid or base to the fermentation mixture. In such cases when ammonia is used to control pH, it also conveniently serves as a nitrogen source. The pH is maintained from about 3.0 to about 8.0, from about 3.5 to about 7.0 or from about 4.0 to about 6.5. The fermentation mixture can optionally be maintained to have a dissolved oxygen content during the course of fermentation to maintain cell growth and to maintain cell metabolism for production of the nutrients. The oxygen concentration of the fermentation medium can be monitored using known methods, such as through the use of an oxygen electrode. Oxygen can be added to the fermentation medium using methods known in the art such as through agitation and aeration of the medium by stirring, shaking or sparging. Fermentation may occur subsequent to enzymatic hydrolysis, or may occur concurrently with enzymatic hydrolysis by SSF. In some aspects of the present disclosure, SSF can keep the sugar levels produced by hydrolysis low thereby reducing potential product inhibition of the hydrolysis enzymes, reducing sugar availability for contaminating microorganisms, and improving the conversion of treated biomass to monosaccharides and/or oligosaccharides.

In some aspects of the present disclosure, the fermentation organism can be adapted to the inhibitors present in the liquefied slurry 134 or the monosaccharide rich stream 135. Although the following focuses on use of yeast in fermentation of sugars, it is to be understood that any organism (e.g., yeast or bacteria) suitable for metabolizing monosaccharide streams of the present disclosure may be utilized in the process of the disclosure. In such aspects, a portion of the liquefied slurry 134 or the monosaccharide rich stream 135 is introduced into a fermentive organism adaptation vessel such that from about 0.5 wt. % to about 25 wt. %, from about 0.5 wt. % to about 10 wt. % or from about 2 wt. % to about 6 wt. % of the monosaccharide fraction in the adaptation vessel is provided by the liquefied slurry or monosaccharide rich stream. A yeast culture is added to the adaptation vessel to grow yeast able to more efficiently transform lignocellulosic hydrolyzate comprising inhibitors to ethanol. Generally, the mass ratio of yeast and monosaccharide fraction introduced into the yeast adaptation vessel is at least about 0.05:1, or at least about 0.1:1. For example, typically the mass ratio of yeast and monosaccharide fraction is from about 0.05:1 to about 0.25:1 and, more typically, from about 0.1:1 to about 0.2:1. Yeast is typically in the form of a solution or slurry of yeast dissolved in or dispersed throughout a suitable liquid medium. For example, in various embodiments, yeast has a total solids content of from about 1 to about 20 wt. %, or from about 5 wt. % to about 15 wt. %. Typically, the yeast-containing liquid medium contains the yeast at a concentration of from about 0.60 g/L to about 150 g/L, or from about 0.80 g/L to about 120 g/L. Supplements may be introduced into yeast adaptation vessel. The supplement is generally in the form of a solution and comprises syrup, cane molasses, beet molasses, water, urea, commercial yeast nutrients, or a combination thereof. Also, in various preferred embodiments, to promote yeast cell growth and adaptation to inhibitors, filtered air may be supplied to the adaptation vessel to provide advantageous oxygen transfer required for yeast growth.

Fermentation products may be recovered using any of various methods known in the art. For instance, fermentation products may be separated from other fermentation components by distillation (e.g., azeotropic distillation), liquid-liquid extraction, solid-liquid extraction, adsorption, gas stripping, membrane evaporation, pervaporation, centrifugation, crystallization, filtration, microfiltration, nanofiltration, ion exchange, or electrodialysis. As a specific example, methanol, ethanol, 1-butanol, or other fermentation products having sufficient volatility, may be recovered from a fermentation mixture by distillation to generate a product stream and a whole stillage stream. In another example, 1-butanol may be isolated from a fermentation mixture using methods known in the art for acetone-butanol-ethanol (“ABE”) fermentations (see for example, Dune, Appl. Microbiol. Biotechnol. 49:639-648 (1998), Groot et al., Method. Biochem. 27:61-75 (1992), and references therein), for instance by solids removal followed by isolation by distillation, liquid-liquid extraction, adsorption, gas stripping, membrane evaporation, or pervaporation. In yet another example, 1,3-propanediol may be isolated from a fermentation mixture by extraction with an organic solvent, distillation, and column chromatography (see U.S. Pat. No. 5,356,812). In yet another example, amino acids may be collected from fermentation mixture by methods such as ion-exchange resin adsorption and/or crystallization. Selection of a suitable separation method for any particular fermentation product may be done by those skilled in the art.

When introducing elements of the present disclosure or the aspects(s) or embodiments(s) thereof, the articles “a”, “an”, “the” and “said” are intended to mean that there are one or more of the elements. The terms “comprising”, “including” and “having” are intended to be inclusive and mean that there may be additional elements other than the listed elements.

This written description enables any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have elements that do not differ from the literal language of the claims, or if they include equivalent elements with insubstantial differences from the literal languages of the claims.

1. A method for hydrolyzing lignocellulosic biomass, the method comprising:
(1) contacting the lignocellulosic biomass with an aqueous acid to form an acid impregnated lignocellulosic biomass having a pH of less than 4 and a total solids content of from about 30 percent by weight to about 70 percent by weight;
(2) contacting the acid impregnated lignocellulosic biomass with steam to achieve a temperature of from about 150° C. to about 250° C. and a pressure of from about 380 kPa gauge to about 3870 kPa gauge;
(3) depressurizing the acid impregnated lignocellulosic biomass to a pressure of from about 20 kPa to about 100 kPa gauge and reducing the temperature of from about 105° C. to about 120° C. to form steam pretreated acid impregnated lignocellulosic biomass and flashed condensate;
(4) extracting the steam pretreated acid impregnated lignocellulosic biomass with a sufficient amount of a chilled aqueous alkaline solution to form (i) an extracted lignocellulosic biomass slurry comprising less than 25 percent by weight total solids and having a temperature of from 30° C. to about 70° C. and a pH of from about 4 to about 6 and (ii) a rich liquor aqueous stream comprising at least 60 percent by weight of the soluble compounds present in the stream pretreated acid impregnated biomass;
(5) dewatering the extracted lignocellulosic biomass slurry to form (i) an extracted dewatered lignocellulosic biomass comprising at least 25 percent by weight total solids and having a substantially uniform pH of from about 4 to about 6 and a substantially uniform temperature of from 30° C. to about 70° C. and (ii) a lean liquor aqueous stream; and
(6) contacting the extracted dewatered lignocellulosic biomass with a first source of enzymes comprising at least cellulase to form a slurry having at least 25 percent by weight solids and hydrolyzing said slurry to form a primary hydrolyzate having a viscosity of less than about 10,000 centipoise and comprising hexose and pentose sugars.

2. The method of claim 1 wherein the steam pretreated acid impregnated lignocellulosic biomass is extracted at weight ratio of liquid to total insoluble compounds of from about 2:1 to about 2.5:1.

3-4. (canceled)

5. The method of claim 1 wherein the extracted lignocellulosic biomass slurry comprises from about 5 to about 22 percent by weight total solids.

6-8. (canceled)

9. The method of claim 1 wherein the total solids content of the extracted dewatered lignocellulosic biomass is from about 25 percent by weight to about 35 percent by weight.

10-17. (canceled)

18. The method of claim 1 wherein the acid impregnated lignocellulosic biomass is extracted by contact with the chilled aqueous alkaline solution in a counter-current diffusion scheme.

19. The method of claim 1 wherein the rich liquor stream comprises inhibitor compounds selected from the group comprising enzymatic inhibitors, fermentation inhibitors, and combinations thereof, and the method further comprises removing at least a portion of the inhibitors from said stream by contact with (i) activated carbon and/or (ii) an ion exchange resin.

20. (canceled)

21. The method of claim 1 further comprising:
(1) cooling at least a portion of the rich liquor stream to a temperature of from about 5° C. to about 20° C. and adjusting the pH of said stream with a base to a pH of from about 4 to about 7 thereby forming an adjusted rich liquor stream;
(2) depressurizing the acid impregnated lignocellulosic biomass in a flash tank; and
(3) recycling at least a portion of the adjusted rich liquor stream to the flash tank to cool and adjust the pH of the acid impregnated lignocellulosic biomass.

22. The method of claim 1 wherein at least a portion of the rich liquor aqueous stream is removed from the process as a xylose-rich sugar solution.

23-24. (canceled)

25. The method of claim 1 further comprising an intermediate depressurization step wherein the acid impregnated lignocellulosic biomass is depressurized to a pressure of from about 140 kPa gauge to about 690 kPa gauge and held for from about 1 minute to about 45 minutes prior to depressurizing to from about 20 kPa to about 100 kPa gauge.

26. The method of claim 1 further comprising cooling the lean liquor stream to a temperature of from about 5° C. to about 20° C. and recycling at least a portion of said stream to the steam pretreated acid impregnated lignocellulosic biomass extraction.

27-31. (canceled)

32. The method of claim 21 wherein the at least a portion of the adjusted rich liquor stream is combined with the primary hydrolyzate.

33. The method of claim 1 further comprising contacting the primary hydrolyzate with a second source of enzymes comprising at least a xylanase in a secondary enzymatic hydrolysis step to form a secondary hydrolyzate enriched in pentose sugar as compared to the primary hydrolyzate.

34. (canceled)

35. The method of claim 33 further comprising fractionating the secondary hydrolyzate form an aqueous stream comprising hexose and pentose sugars in solution and a solid residue stream, wherein the aqueous stream is enriched in hexose and pentose sugars as compared to the solid residue stream.

36-38. (canceled)

39. The method of claim 1 wherein the enzyme dosage required to achieve a given glucose yield based on cellulose content of the extracted dewatered lignocellulosic biomass is at least about 10% less than the enzyme dosage required a similar glucose yield for dewatered lignocellulosic biomass prepared by a comparable process, differing with respect to the absence of the extracting and dewatering steps.

40. The method of claim 1 wherein the enzyme dosage required to achieve a given glucose yield based on cellulose content of the extracted dewatered lignocellulosic biomass as compared to the enzyme dosage required a similar glucose yield for dewatered lignocellulosic biomass prepared by a comparable process, differing with respect to the absence of the extracting and dewatering steps, is reduced by about 25% at a 80% glucose yield.

41. A method for hydrolyzing lignocellulosic biomass, the method comprising:
(1) contacting the lignocellulosic biomass with an aqueous acid to form an acid impregnated lignocellulosic biomass having a pH of less than 4 and a total solids content of from about 40 percent by weight to about 70 percent by weight;
(2) contacting the acid impregnated lignocellulosic biomass with steam to achieve a temperature of from about 150° C. to about 250° C. and a pressure of from about 380 kPa gauge to about 3870 kPa;
(3) depressurizing the acid impregnated lignocellulosic biomass in a flash tank to a pressure of from about 20 kPa to about 35 kPa gauge and reducing the temperature to from about 105° C. to about 108° C. to form steam pretreated acid impregnated lignocellulosic biomass and flashed condensate;
(4) combining alkaline chilled cooling water with the steam pretreated acid impregnated lignocellulosic biomass in the flash tank and discharging the flash tank contents to a dynamic mixer;
(5) adding additional alkaline chilled cooling water to the dynamic mixer and admixing the alkaline chilled cooling water and the steam pretreated acid impregnated lignocellulosic biomass in the dynamic mixer to produce adjusted steam pretreated acid impregnated lignocellulosic biomass, wherein the amount and pH of the alkaline chilled cooling water is sufficient to provide an adjusted steam pretreated acid impregnated lignocellulosic biomass having a total solid content of at least 30 percent by weight, an average temperature of from about 30° C. to about 70° C., an average pH of from about 4 to about 6, and a viscosity of greater than about 20,000 centipoise; and
(6) contacting the adjusted steam pretreated acid impregnated lignocellulosic biomass with a first source of enzymes comprising at least cellulase in a primary enzymatic hydrolysis step to form a primary hydrolyzate having a viscosity of less than about 10,000 centipoise and comprising hexose and pentose sugars, and wherein the total solids content of the adjusted steam pretreated acid impregnated lignocellulosic biomass after contact with the first source of enzymes at least 25 percent by weight.

42. The method of claim 41 wherein the acid impregnated lignocellulosic biomass is depressurized in the flash tank to a pressure about 20 kPa gauge and the temperature is reduced to about 105° C.

43-49. (canceled)

50. The method of claim 41 further comprising an intermediate depressurization step wherein the acid impregnated lignocellulosic biomass is depressurized to a pressure of from about 140 kPa gauge to about 690 kPa gauge and held for from about 1 minute to about 45 minutes prior to depressurizing to from about 20 kPa to about 35 kPa gauge.

51. The method of claim 41 further comprising contacting the primary hydrolyzate with a second source of enzymes comprising at least a xylanase in a secondary enzymatic hydrolysis step to form a secondary hydrolyzate enriched in pentose sugar as compared to the primary hydrolyzate.

52. (canceled)

53. The method of claim 51 further comprising fractionating the secondary hydrolyzate to form an aqueous stream comprising hexose and pentose sugars in solution and a solid residue stream, wherein the aqueous stream is enriched in hexose and pentose sugars as compared to the solid residue stream.

54-57. (canceled)
